(19) United States
(12) Patent Application Publication
(10) Pub. No.: US 2017/0173262 A1
(43) Pub. Date: Jun. 22, 2017

(54) MEDICAL SYSTEMS, DEVICES AND METHODS

(71) Applicant: François Paul VELTZ, CANNES (FR)

(72) Inventor: François Paul VELTZ, CANNES (FR)

(21) Appl. No.: 15/446,015

(22) Filed: Mar. 1, 2017

(51) Int. Cl. A61M 5/172; A61B 5/00; A61B 5/145

(52) U.S. Cl. CPC A61M 5/1723; A61B 5/14532; A61B 5/1451; A61B 5/0004; A61B 5/4839; A61M 2230/201; A61M 2205/502; A61M 2205/3592; A61M 2205/80; A61M 2205/505; G06F 21/575

(57) ABSTRACT

There is disclosed a medical system comprising one or more sensors associated with one or more actuators. Various embodiments describe sensors and/or actuators, logic circuits, user interfaces, association schemes, communication schemes, security schemes, cryptographic schemes, medical management rules, social mechanisms, energy management schemes, time and/or space schemes, body analytes and/or biomarkers, blood glucose and/or interstitial glucose sensors, drug delivery devices, continuous glucose monitoring devices, as well as flash glucose monitoring devices. Methods, software and other hardware aspects are described.

### TECHNICAL FIELD

This document relates to the field of medical systems, devices and methods. More particularly, there are described systems, devices and methods to handle diabetes.

### BACKGROUND

Diabetes is a serious medical condition. A child with type 1 diabetes is endangered by hypoglycemia (low glucose concentration value requiring the intake of carbohydrates) and hyperglycemia (high glucose concentration value requiring the injection of insulin).

During the night, risks are considerably amplified. In particular, the risk of the occurrence of a severe hypoglycemia, which can remain undetected for hours, can lead parents to wake up in the middle of the night to check glucose concentration value of their child. A finger prick can determine blood glucose (BG) values. In itself, a BG measurement can wake up the child. If a low BG value is measured, it is required to wake up the child for the intake of carbohydrates (e.g. sugar drinks). As a result, these manual interventions are detrimental to the quality of life of both child and parents, not even talking of long term consequences of diabetes on the health of the child.

Existing “continuous” or “flash” glucose monitoring devices can be used to monitor blood or interstitial glucose values during the night and to raise alarms if applicable. Yet these devices generally are insufficient. Among other drawbacks, these devices are invasive (the subcutaneous sensor can damage the skin, even if minimally-invasive), generally require additional devices (e.g. a display device and a data transmitter in addition to the glucose sensor), can sometimes require calibration (i.e. standard finger prick) and can be costly. As a result, these monitoring devices hardly can be used permanently.

Existing medical systems, devices and methods to manage diabetes present limitations.

There is a need for advanced medical systems devices and methods to monitor and manage the health condition a patient, for example of a child with diabetes during the night.

### SUMMARY

There is disclosed a medical system comprising one or more sensors associated with one or more actuators. Various embodiments describe sensors and/or actuators, logic circuits, user interfaces, association schemes, communication schemes, security schemes, cryptographic schemes, medical management rules, social mechanisms, energy management schemes, time and/or space schemes, body analytes and/or biomarkers, blood glucose and/or interstitial glucose sensors, drug delivery devices, continuous glucose monitoring devices, as well as flash glucose monitoring devices. Methods, software and other hardware aspects are described.

There is disclosed a medical system comprising one or more sensors associated with one or more actuators. The medical system can comprise one or more medical devices, for example connected medical devices. A medical device or the medical system can comprise sensors and/or actuators.

In an embodiment, the medical system further comprises one or more logic circuits configured to control and/or to interact with one or more of said sensors and/or actuators. Logic circuits (i.e. hardware) embody (e.g. “realize” or “implement”) software. The relationship can be unidirectional (“control”, e.g. in one of the two directions) or can be bidirectional (“interaction”, e.g. with feedback-loop, with feedforward mechanisms, etc)

In an embodiment, the medical system further comprises one or more user interfaces. The interface can be a graphical User Interface (U.I.), in 2D (display screen) and/or in 3D (e.g. augmented and/or virtual reality), with or without haptic input and/or output devices. The UI also can comprise or be performed by audio (sounds, music, etc), vibrations, odors or others (nervous influx, electrical signal, etc).

In an embodiment, parts of the medical system are arranged and/or configured according to association schemes. Subparts of the medical system can be (e.g. physically) arranged and/or (e.g. logically) configured (or adapted) according to different schemes.

In an embodiment, the medical system or parts thereof are arranged and/or configured according to one or more communication schemes. Various communications means (e.g. Wi-Fi, Bluetooth, etc.), protocols, modulations (e.g. CDMA), medium/media (e.g. wired/wireless) or data transport schemes can be used.

In an embodiment, the medical system or parts thereof are arranged and/or configured according to one or more security schemes. Security schemes comprise a Physically Unclonable Function and/or a challenge-response test and/or a True Random Number Generator.

In an embodiment, the medical system or parts thereof are arranged and/or configured according to one or more cryptographic schemes. Cryptographic schemes comprise a Quantum Key Distribution mechanism and/or post-quantum cryptography and/or quantum-safe cryptography and/or crypto-ledger and/or one or more smart contracts configured to control or influence operations of the medical device and/or communications thereof.

In an embodiment, the medical system, parts thereof and/or the control thereof are arranged and/or configured according to one or more medical management rules. Medical management rules can be specific to/for particular medical conditions, for example for diabetes. Some rules can be FDA-regulated. Some others may not be (private use).

In an embodiment, the medical system, parts thereof and/or the control thereof are arranged and/or configured according to one or more social mechanisms. In an embodiment, the medical system, parts thereof and/or the control thereof are arranged and/or configured according to one or more energy management schemes.

In an embodiment, the medical system, parts thereof and/or the control thereof are arranged and/or configured according to one or more time and/or space schemes. Dimensions of sensors and/or actuators can be different (e.g. macro or micro-scales).

In an embodiment, at least one sensor determines the concentration of an analyte and/or of a biomarker.

In an embodiment, at least one sensor is minimally-invasive or non-invasive.

In an embodiment, at least one sensor and/or actuator are implementable.

In an embodiment, the medical system further comprises a contact lens and/or a spectrometer and/or a drone and/or a wearable computer. Said macro-objects can embed said sensors and/or actuators.

In an embodiment, the analyte is blood and/or interstitial glucose. Along/aside glucose, many other analytes can be measured. Blood glucose (BG) values correspond to “capillary” glucose (which can be inferior from plasma or arterial glucose levels by up to 10%). A “glucometer” is a medical device (i.e. approved by the Food and Drug Administration or “FDA”) which provides one BG value at one single point in time with a sample of blood (finger prick).

In an embodiment, at least one actuator is a drug delivery device. In particular, the actuator can be an insulin pump.

In an embodiment, the drug is insulin. Along/aside insulin, many other drugs can be injected or otherwise be made available or accessible.

In an embodiment, the medical system further comprises a Continuous Glucose Monitoring sensor. The sensor can be part of a CGM device. A “continuous glucose monitoring” (CGM) system is a medical device which comprises a sensor with subcutaneous insertion (generally configured to remain in place during 15 days), a data transmitter generally mounted on top of said sensor and a display device (with local or distant processing capabilities). A CGM system provides continuous BG values (a plurality of measures over time). Some models also provide predicted values or trends in BG values. A CGM device is considered to be an “invasive” system (the size of the subcutaneous sensor is significant and leads to skin damages).

In an embodiment, the medical system further comprises a “flash glucose monitoring device” (FGM) associated with an electronic circuit configured to receive and/or send data to/from said flash glucose monitoring device and to/from a remote computer device such as a smartphone. A flash glucose monitoring (FGM) device is a medical device which provides BG values “on demand” or “upon (manual) request” (in particular by Near Field Communication or “NFC”). A FGM device is considered to be a “minimally-invasive” system, as the size of the FGM sensor is significantly smaller than the one of a CGM. Such a system is reported as being accurate, stable and consistent over 14 days without the need for finger prick calibration.

### BRIEF DESCRIPTION OF DRAWINGS

Particular embodiments of the present invention will now be described with reference to the accompanying drawings in which references denote similar elements.

Wherein applicable, the enclosed drawings are copyrighted.

FIG. 1 provides an overview of described embodiments;

FIG. 2 shows a specific embodiment of the invention;

FIG. 3 shows another example of an embodiment of the invention;

FIG. 4 illustrates association schemes of sensors and/or actuators according to embodiments of the invention.

### DETAILED DESCRIPTION

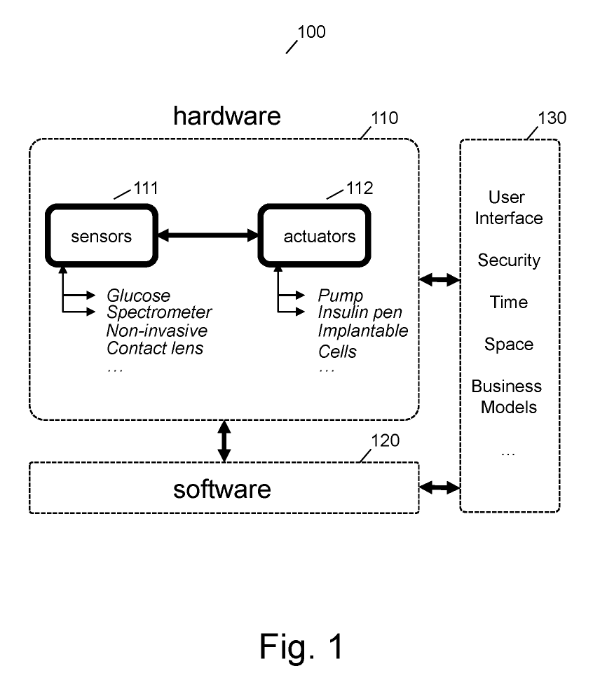

FIG. 1 provides an overview of described embodiments.

FIG. 1 shows aspects of a medical system 100 comprising hardware 110 and software 120. Hardware 110 and/or software 120 can be controlled by (and/or can control) various elements 130 (e.g. user interfaces, security schemes, time and/or space schemes, etc).

Hardware 110 comprises one or more sensors 111 associated with one or more actuators 112. Software 120 can correspond to one or more logic circuits configured to control and/or to interact with one or more of said sensors and/or actuators.

Hardware 110 and/or software 120 can be controlled or controllable by one or more user interfaces. The medical system 100 (110, 120, 130) can comprise/implement or be associated by/with association schemes, communication schemes, security schemes, cryptographic schemes, medical management rules, social mechanisms, energy management schemes, time and/or space schemes, various body analytes and/or biomarkers. Various business models translatable into technical features can interact with the medical system.

The medical system according to the invention can comprise one or more sensors associated with one or more actuators.

### Sensors

The medical system can comprise one or more medical devices, for example connected medical devices. A medical device or the medical system can comprise sensors and/or actuators.

Embodiments of the invention can comprise one or more sensors, selected from the group comprising a geophone, hydrophone, microphone, position sensor, air-fuel ratio meter, blind spot monitor, crankshaft position sensor, curb feeler, defect detector, temperature sensor, ECT sensor temperature sensor, Hall effect sensor, pressure sensor, flow sensor, oxygen sensor, parking sensor, speedometer, speed sensor, reluctance sensor, Breathalyzer, Carbon dioxide sensor, Carbon monoxide detector, Catalytic bead sensor, Chemical field-effect transistor, Electrochemical gas sensor, Electronic nose, Electrolyte-insulator-semiconductor sensor, Fluorescent chloride sensor, Holographic sensor, Hydrocarbon dew point analyzer, Hydrogen sensor, Hydrogen sulfide sensor, Infrared point sensor, Ion-selective electrode, Nondispersive infrared sensor, Microwave chemistry sensor, Nitrogen oxide sensor, Olfactometer, Optode, Oxygen sensor, Ozone monitor, Pellistor, pH glass electrode, Potentiometric sensor, Redox electrode, Zinc oxide nanorod sensor, Current sensor, Electroscope, Galvanometer, Hall effect sensor, Hall probe, Magnetic anomaly detector, Magnetometer, microelectromechanical systems (MEMS), magnetic field sensor, Metal detector, Planar Hall sensor, Radio direction finder, Voltage detector, Actinometer, Bedwetting alarm, Ceilometer, Dew warning, Electrochemical gas sensor, Fish counter, Frequency domain sensor, Gas detector, Hook gauge evaporimeter, Humistor, Hygrometer, Leaf sensor, Pyranometer, Pyrgeometer, Psychrometer, air flow meter, liquid flow meter, anemometer, mass flow sensor, Water meter, Bubble chamber, Geiger counter, neutron detection, Particle detector, Scintillation counter, Scintillator, Wire chamber, Air speed indicator, Altimeter, Attitude indicator, Depth gauge, Fluxgate compass, Gyroscope, Inertial navigation system, Inertial reference unit, Magnetic compass, MHD sensor, Ring laser gyroscope, Turn coordinator, Variometer, Vibrating structure gyroscope, Yaw rate sensor, Accelerometer, Auxanometer, Capacitive displacement sensor, Capacitive sensing, Free fall sensor, Gravimeter, Gyroscopic sensor, Inclinometer, Integrated circuit piezoelectric sensor, Laser rangefinder, Laser surface velocimeter, LIDAR, Linear encoder, Linear variable differential transformer (LVDT), Liquid capacitive inclinometers, Odometer, Photoelectric sensor, Piezoelectric accelerometer, Position sensor, Rate sensor, Rotary encoder, Rotary variable differential transformer, Tilt sensor, Tachometer, Ultrasonic thickness gauge, Variable reluctance sensor, Velocity receiver, Charge-coupled device, Colorimeter, Contact image sensor, Electro-optical sensor, Flame detector, Infra-red sensor, Kinetic inductance detector, LED, light sensor, Light-addressable potentiometric sensor, Nichols radiometer, Fiber optic sensor, Optical position sensor, Photodetector, Photodiode, Photomultiplier tubes, Phototransistor, Photoelectric sensor, Photoionization detector, Photomultiplier, Photoresistor, Photoswitch, Phototube, Scintillometer, Shack-Hartmann sensor, Single-photon avalanche diode, Superconducting nanowire single-photon detector, Transition edge sensor, Visible light photon counter, Wavefront sensor, Barograph, Barometer, Boost gauge, Bourdon gauge, Hot filament ionization gauge, Ionization gauge, Oscillating U-tube, Permanent Downhole Gauge, Piezometer, Pirani gauge, Pressure sensor, Pressure gauge, Tactile sensor, Time pressure gauge, Bhangmeter, Hydrometer, Force gauge, Level sensor, Load cell sensor, Magnetic level gauge, Nuclear density gauge, Piezoelectric sensor, Strain gauge, Torque sensor, Viscometer, Bolometer, Bimetallic strip, calorimeter, Exhaust gas temperature gauge, Flame detection, Gardon gauge, Golay cell, Heat flux sensor, Infrared thermometer, Microbolometer, Microwave radiometer, Net radiometer, Quartz thermometer, Resistance temperature detector, Resistance thermometer, Silicon bandgap temperature sensor, Special sensor microwave/imager, Temperature gauge, Thermistor, Thermocouple, Thermometer, Pyrometer, Alarm sensor, Doppler radar, Motion detector, Occupancy sensor, Proximity sensor, Passive infrared sensor, Reed switch, Stud finder, Triangulation sensor, Touch switch, Wired glove, Active pixel sensor, Back-illuminated sensor, Biochip, Biosensor, Capacitance probe, Catadioptric sensor, Carbon paste electrode, Digital sensor, Displacement receiver, Electromechanical film, Electro-optical sensor, Fabry-Pérot interferometer, Fisheries acoustics, Image sensor, Image sensor format, Inductive sensor, Lab-on-a-chip, Leaf sensor, Machine vision, Micro-sensor arrays, Photoelasticity, Quantum sensor, RADAR, Ground-penetrating radar, Synthetic aperture radar, Radar tracker, SONAR, Staring array, Transducer, Ultrasonic sensor, Video sensor, Visual sensor network, Wheatstone bridge, Wireless sensor network, Actigraphy, Analog image processing, Atomic force microscopy, Catadioptric sensor, Chemoreceptor, Compressive sensing, Cryogenic particle detectors, Dew warning, Diffusion tensor imaging, Digital holography, Electronic tongue, Fine Guidance Sensor, Flat panel detector, Functional magnetic resonance imaging, Glass break detector, Heartbeat sensor, Hyperspectral sensor, biosensor, Interferometric Reflectance Imaging Sensor, Laser beam profiler, Millimeter wave scanner, Magnetic resonance imaging, Moire deflectometry, Molecular sensor, Nanosensor, Nano-tetherball Sensor, Omnidirectional camera, Optical coherence tomography, Positron emission tomography, Push broom scanner, sensitive air-conductivity sensor, Range imaging, Scanning SQUID microscope, Single-Photon Emission Computed Tomography, Superconducting quantum interference device, SSIES, SSMIS, Structured-light 3D scanner, Superconducting nanowire single-photon detector, Thin-film thickness monitor, Time-of-flight camera, TriDAR, etc.

An accelerometer can be used to recognize and monitor body posture, such as sitting, kneeling, crawling, laying, standing, walking and running. Such ability can be essential to many applications, including virtual reality, healthcare, sports and electronic games. The accelerometer-based posture monitoring for BANs typically consists of 3-axis accelerometers (or tri-axial accelerometers) which can be placed on some strategic locations on a human body. They can also be used to measure the vibration, as well as acceleration due to the gravity. A gyroscope can be used for measuring or maintaining orientation, based on the principle of conservation of angular momentum. Gyroscopes can be used together with accelerometers for physical movement monitoring. One or more accelerometers can quantify the physiological state of the patient.

The medical system may measure, calculate, or use a plurality of other physiological metrics in addition to, or in place of, the user's step count. These include, but are not limited to, caloric energy expenditure, floors climbed or descended, heart rate, heart rate variability, heart rate recovery, location and/or heading (e.g., through GPS), elevation, ambulatory speed and/or distance traveled, swimming lap count, bicycle distance and/or speed, blood pressure, blood glucose, skin conduction, skin and/or body temperature, electromyography data, electroencephalographic data, weight, body fat, and respiration rate. Some of this data may be provided to the biometric monitoring device from an external source, e.g., the user may input their height, weight, and stride in a user profile on a fitness-tracking website and such information may then be communicated to the biometric monitoring device via the I/O interface and used to evaluate, in tandem with data measured by the biometric sensors, the distance traveled or calories burned of the user.

Blood glucose (BG), also called blood sugar, can be the amount of glucose circulating in the blood. Traditionally, glucose measurements are done by lancing a finger and extracting a drop of blood, which is applied to a test strip that includes chemicals sensitive to the glucose in the blood sample. An optical or electrochemical detector (glucometer) can be used to analyze the blood sample and can give a numerical glucose reading. Recently, non-invasive glucose measuring devices that monitor BG through infrared technology and optical sensing have become available;

A blood pressure sensor can be a non-invasive sensor designed to measure systolic and diastolic human blood pressure utilizing the oscillometric technique;

A CO2 gas sensor measures gaseous carbon dioxide levels to monitor changes in CO2 levels as well as to monitor oxygen concentration during human respiration;

ECG sensor: ECG is a graphic record of the heart's electrical activity. Healthcare providers use it to help diagnose a heart disease as well as to monitor how well different heart medications are working. In order to obtain an ECG signal, several electrodes can be attached at specific sites on the skin (e.g., arms, and chest) and the potential differences between these electrodes are measured;

An EEG sensor measures the electrical activity within the brain by attaching small electrodes to the human's scalp at multiple locations. Then, information of the brain's electrical activities sensed by the electrodes can be forwarded to an amplifier for producing a pattern of tracings.

Synchronous electrical activities in different brain regions are generally assumed to imply functional relationships between these regions. In a hospital, the patient may be asked to breathe deeply or to look at a flashing light during the recording of EEG;

An EMG sensor measures electrical signals produced by muscles during contractions or at rest. Nerve conduction studies are often done together with measuring the electrical activity in muscles, since nerves control the muscles in the body by electrical signals (impulses) and these impulses make the muscles react in specific ways. Nerve and muscle disorders cause the muscles to react in abnormal ways;

A pulse oximetry measures oxygen saturation using a non-invasive probe. A small clip with a sensor is attached to the person's finger, earlobe, or toe. The sensor gives off a light signal that passes through the skin. According to the light absorption of oxygenated hemoglobin and total hemoglobin in arterial blood, the measurement is expressed as a ratio of oxygenated hemoglobin to the total amount of hemoglobin;

Humidity and temperature sensors can be used for measuring the temperature of the human body and/or the humidity of the immediate environment around a person. An alarm signal can be issued if a certain amount of changes are measured; and

Imaging sensors (camera, video cameras, etc): by computer vision, data can be extracted or inferred from data streams. An embedded video camera can monitor the state of the skin at sensor insertion (e.g. within the glucose sensor).

Flow sensors can be used (e.g. at pump delivery outlet and/or at the tip of cannula and/or within the body/skin). Static (e.g. volumes) and/or dynamic data can be measured (e.g. speed, kinetics, flow etc).

A sensor can comprise a lab-on-a-chip. A sensor can comprise a DNA chip.

Contextual sensors can be sensors which can be present in the environment (RFID tags providing GPS information, nutritional values of meals, etc.) or worn by the patient. Some may also be implemented in the body of the user. These sensors can assess the current lighting conditions (night, dark, sunny, etc.), can probabilistically assess or classify the ambient audio level (restaurant, nightclub, working environment, sleeping room, outdoor activities assessed by the presence of wind sounds for example, indoor activities assessed by the presence of particular acoustic responses or audio signals such as music for example), can determine a geographical location such as a GPS sensor for example, can perform human face detection (the device can continuously monitor this parameter in order to provide an appropriate response upon the detection of the face of the user looking at the medical infusion device for example), can evaluate the distance to the eye of the user—a user looking at the medical device. Some sensors can detect the breath of the user (when the user stands very close to the device, for example, during the night). The sensors mentioned above can be combined. For example, the proximity of the user face can be confirmed by the detection of the breath of the user in the proximity of the sensor.

Sensors can be interoperable. One or more sensors can be interdependent, forming a dependency scheme. Some others can be identified as independent. A graph can allow detection of super nodes, i.e. active regulation entries.

Contextual and body sensors can be combined together.

Access to sensors can be remote, via APIs for example. The Body Area Network of sensors can be adaptive, reconfigurable depending on activated sensors. The BNA can comprise sound amplifiers, anemometers to quantify breathing, cameras, gyroscopes, etc

The emotions of the patient while sleeping can be estimated (in the voice signal if applicable, movements of the face, etc) and further remotely communicated (for example to parents). Eye-tracking i.e. movement of the eyes of the patient can be measured or estimated. Geolocation can be used, for example to trigger particular diabetes management rules. Gestures can be quantified, thanks to the use of one or more accelerometers. One or more microphones can be used (to estimate the patient distress if applicable). Selective microphones can be used. Ear buds can monitor heart rates.

Food scanners can for example communicate how many and what kind of ingredients, how many allergens, toxins, how many carbohydrates a given food actually contains.

Shapes (of sensors and/or injectors) can be complex. Shapes of the sensor and/or the injection device can optimize data capture and/or drug delivery. Some shapes can be advantageously employed, for example, butterfly-shaped, round, square, or rectangular. For example, shapes in spirals (two-dimensional spiral or three-dimensional spiral) can increase the surface in contact with blood analyte, while presenting different skin penetration profiles. Advantageous shapes (for one or more sensors or injection devices or tips) can comprise one or more of a dihedral angle or solid angle, a cube, a cuboid, a parallelepiped, a tetrahedron, a pyramid, a prism, an octahedron, a dodecahedron, an icosahedron, a cone, a cylinder, a sphere, a spheroid, an ellipsoid, a paraboloid, an hyperboloid. Other shapes are possible. A specific complex advantageous spiral can comprise one or more parts of a Archimedean spiral, Cornu spiral, Fermat's spiral, hyperbolic spiral, logarithmic spiral, spiral of Theodorus, Fibonacci Spiral (golden spiral) for example.

Patterns (for sensors and/or injectors) can be complex. Such patterns can be used for optimal or improved blood analyte sampling, to determine the structure (e.g. layers) of reagent coatings, to arrange gaps or apertures in injectors (one or more injections devices or structures or cannulas). Patterns can be symmetrical or asymmetrical. Patterns can comprise one or more of a tree, a fractal structure (e.g. to increase contact surfaces), a spiral, a flow, a meander, a wave, a dunes, a bubble, foam, a crack, a spot or a stripe. Geometrical shapes can be use convex polyhedron, geodesic domes etc. Patterns can comprise tessellations (patterns formed by repeating tiles all over a surface). Groups of tilings can include wax cells (such as those in honeycomb). Tiles can be overlapping. Patterns can use regularly repeating three-dimensional arrays (e.g. crystal structure, Bravais lattices for lattice systems in three-dimensional space). Crystal shapes can be cube-shaped crystals. Other forms include but are not limited to arrays, tilings, pavements, reticulate structures, etc. Textile patterns are also possible (e.g. end-on-end, pin stripes, rain pattern, toile, etc. Surfaces can comprise one or more of a minimal surface, a ruled surface, a non-orientable surface, a quadrics, a pseudospherical surfaces or an algebraic surface. Some patterns can be controllable (e.g. configurable at start or dynamically, evolve over time, etc).

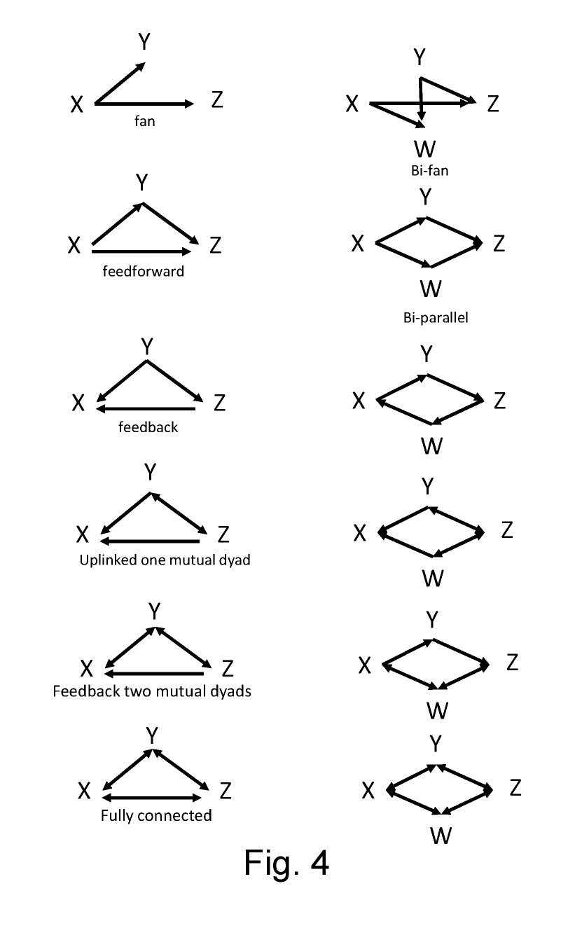

Sensors for example can be arranged in array, data fusion, in a grid, in one or more interconnected graphs (discussed in FIG. 4)

### Actuators

An actuator can be an insulin pump. An insulin pump can be a peristaltic pump. An insulin pump can be a pneumatic pump. An insulin pump can use one or more springs. An insulin pump can use one or more dynamos. In an embodiment, an insulin pump uses a technology similar to ink-printing (e.g. droplets). In an embodiment, the pump can deliver both insulin and glucagon (or the like). In an embodiment, the pump can comprise slots or cartridges or supports for (small) reservoirs for insulin and/or glucagon. For example a diabetes management scheme can comprise the sequence: during the night a pump is charged with glucagon in order to counteract an hypoglycemia if any (while basal insulin is delivered by pen for the night); during the day: the pump is charged with insulin. In an embodiment, other human/natural or artificial/synthesized hormones can be used(e.g. somatostatin but also one or more of prolactin, adrenocorticotropic hormone (ACTH), vasopressin, oxytocin, atrial-natriuretic peptide, atrial natriuretic factor, cholecystokinin, gastrin, leptin, etc).

Other embodiments are now described. The extension can be hydrophobic. The extension can comprise an inflatable part to protect it from clothes. The injection can use inkjet-like technologies.

In some embodiments, the system according to the invention can be coupled or combined or integrated with an insulin pump and/or an insulin roller and/or an insulin patch provided with micro-needles. In some embodiments, micro-fluidics can be used (e.g. patch pump, insulin patch, tattoo comprising elastic or otherwise flexible electronics).

In an embodiment, a glucose sensor can be partly self repairable. MEMS can be used. Synthetic biology can be used. DIY biology can be used. DNA synthesis can be used. CRISPR (Clustered regularly-interspaced short palindromic repeats) technology can be used. For example, the sensitivity or even the chemistry of an analyte sensor can be personalized.

A drug delivery device and/or an analyte sensor inserted under the skin can use one or more shock absorbers (serial or parallel arrangements), in order to smooth the impact of the movements of the patient. Shock absorption can be passive but also active if not reactive or adaptative (MEMS or actuators can counterbalance mechanical constraints). A contrario skin or tissue massage (facilitating the diffusion of drug and/or analyte) can be used with similar electro-mechanical miniaturized devices.

In an embodiment, an artificial tissue, attached to the skin of the user can store one or more drugs to mitigate infusion (e.g. optimize insulin depots). The tissue can be bio-compatible. In an embodiment, the tissue can comprise flexible electronics. Digital tattoos can be used in combination with described embodiments.

In some embodiments, regulation can closed-loop (“artificial” or “automated” pancreas) or open-loop (with user intervention, e.g. at least confirmation). Artificial pancreas can use bio-inspired subsystems, encapsulated Langerlans cells, stem cells, bio-machines, bio-mechatronics, bio-plastics, bio-polymer, biochips, bionics, biosensors.

The injection of insulin can be performed with a pen and needle, or with pumped air, or via a medicament, or injected by micro-drone. A companion robot (for example a humanoid robot) can be used, for bringing required devices and/or to assist injections.

Insulin “depots” under the skin after a bolus injection depend on many parameters and in particular can vary from person to person and also from injection site to injection site. It is generally not equivalent to inject 1 times 20 units than 4 times 5 units (the volume is not likely to be the same and the dynamics for diffusion of insulin into the blood stream can be modified, in turn changing glycemic response). A specific approach to optimize bolus injection is to use a sprinkler cannula, i.e. a cannula subcutaneously inserted into the body which comprises a plurality of “holes” or “gates” or “pathways” or “apertures” or “perforations” to infuse the drug at different depths. The geometry of holes can be configured to facilitate diffusion profiles (i.e. number of gates, shapes, diameters, distribution in space around the cannula, along the depth axis etc). The infusion can be directed—or at least favored into—one particular of space. In an embodiment, the pathways can be controlled (mechanical and/or chemical and/or electronic controls), so that to allow dynamic control (for example coupled with imaging devices estimating the drug depot).

In some embodiments, a manual or automated prink can be avoided, as well as the need for a subcutaneous (or intravenous) sensor. For example, there can be used a needle-free drawing device. Such a device can be provided with a negative-pressure chamber, for example with a membrane sealing an aperture. A micro-particle can be shot (e.g. by release of gas and/or electromagnetic railgun and/or pneumatic accelerator) and further can pierce the aperture membrane and penetrate adjacent dermal tissue. The micro-emergence or droplet of blood can further be drawn into the negative pressure barrel. Vacuum also can be used, in an alternative or in combination. The micro-particle can comprise an agglomeration of nanoparticles bound together with a biodegradable matrix (e.g. the nanoparticles can comprise nano-sized gold particles and a biodegradable matrix can comprise polylactic-co-glycolic acid). In some embodiments, the micro-particle can comprise a micro-droplet of liquid and/or a medically therapeutic substance. Such a needle-free device can be implemented in a smart watch and for example coupled with a glucometer or other blood analysis device.

### Software

In an embodiment, the system according to the invention can further comprise one or more logic circuits configured to control and/or to interact with one or more of said sensors and/or actuators.

Logic circuits (i.e. hardware) embody (e.g. “realize” or “implement”) software. The relationship can be unidirectional (“control”, e.g. in one of the two directions) or can be bidirectional (“interaction”, e.g. with feedback-loop, with feedforward mechanisms, etc)

The term “processor” designates one or more of a general-purpose microprocessor (e.g., a central processing unit (CPU)), a graphics processing unit (GPU), a microcontroller, a Digital Signal Processor (DSP), an Application Specific Integrated Circuit (ASIC), a Field Programmable Gate Array (FPGA), a Programmable Logic Device (PLD), a controller, a state machine, gated logic, discrete hardware components, or any other suitable entity that can perform calculations or other manipulations of information. A processor can be multi-core or many-core.

Computing processes and/or threads can be discriminated, for example according to their nature. A grade or score for example can be associated with a degree of medical priority. Priority schemes can be complex in the very details. For example, the granularity of computing and/or storage requirements can depend or be a function of or adjust latency, bandwidth, CPU core (e.g. load, parking, stability, etc), caching, performance, etc.

A watchdog or daemon can monitor the appropriate functions of the system and raise alarms if applicable.

Algorithmic complexity of code can be estimated. The controllability of loosely-coupled sensors subsystems can be monitored.

Data scraping can be used. Data scraping designates the operation to escape data from a closed system without data export capabilities (by physical and/or logical if not by intentional limitation). It can use techniques such as image acquisition and Optical Character Recognition (OCR), video acquisition and pattern matching, voice recognition, big data enriching captured data etc. Data scraping can be used to export data out of a proprietary reader having access to a proprietary glucose sensor. Even if data can be encrypted when stored or during transport, at some point data will be deciphered and analogic signal will be available (e.g. visual signal or display), offering an opportunity for data scraping.

The source code of a pump connected to the sensor and extension according to the invention can be hardened (i.e. in binary form and encrypted, possibly obfuscated).

The software at least partly can be executed in a virtual machine (e.g. using sandboxed applications and/or threads).

In some embodiments, the source code and/or the binary code executed by any one part of the system according to the invention, including any described development or embodiment, can be obfuscated (passively or actively i.e. with active defense in case of a detection of code analysis or reverse engineering attempt).

### Hardware

Computing and storage resources of the medical system can be locally accessed and/or accessed from locations in the “cloud” (in one or more servers accessible by one or more communication channels). A local medical device, e.g. an infusion device, can comprise the core medical features (drug reservoir, source of energy, motor or equivalent, injection and/or withdrawal devices for continuous or intermittent monitoring for example). It may further comprise communication capabilities (according to continuous or episodic or intermittent or even opportunistic modes). In other words, a computer (processor) and storage (memory unit) can be remotely accessed. According to this view, the display embodiment can remain mostly unchanged. The rendering of the data to be displayed may be handled in the cloud.

The local display device can then act as a display device (i.e. the images being communicated to the local display device can be uncompressed and do not need local processing steps).

In an embodiment, the extension is hot-swappable (hotplug is possible, reverting to a FGM from a CGM or the opposite)

In an embodiment, the extension can host one or more media, for example stored in a micro-SD card (with video or audio tutorials accessible to a computer nearby, in order to help a patient to use the system according to the invention.

In some embodiments, the extension can use one or more of a memristor and/or a MEMS.

In some embodiments, the architecture can be modular (different parts can be connected, and individually replaced). In an embodiment, each part of the architecture can broadcast service messages and the global system can be coordinated. In some embodiments, some parts can be 3D printed if not bio-printed. As architectures for a network of connected devices, a P2P model can be implemented (or a P3P model in some embodiments)

Association or pairing between apps can use QR codes, or barcodes, or tokens, or communication protocols, with or without the intervention of a user. The disconnection of one or more parts can trigger an alarm.

### User interfaces

Embodiments of the invention can comprise one or more user interfaces (UI). The medical system and/or components thereof can be provided with UI or I/O (input/output) interfaces, providing control endpoints.

The UI can be a graphical User Interface (U.I.), in 2D (display screen) and/or in 3D (e.g. augmented and/or virtual reality), with or without haptic input and/or output devices. The UI also can comprise or be performed by audio (sounds, music, etc), vibrations, odors or others (nervous influx, electrical signal, etc).

User interfaces (man-machine interfaces and/or man-man interfaces) can use voice commands or text-to-speech or speech-to-text steps or technologies.

A diversity of display devices can be used to restitute BG values, trends or other information related to diabetes management. For example, a retinal laser display can be used. One or more pico-projectors can be used, for opportunistic display of information on surroundings surfaces in the environment.

A display may be integrated in head-mounted displays. A head-mounted display can be a display device, worn on the head, which can have a small display optic in front of one (monocular) or each eye (binocular). A typical head-mounted display can have either one or two small displays with lenses and semi-transparent minors embedded in a helmet, eye-glasses (also known as data glasses) or visor. The display units are miniaturized and may include CRT, LCDs, or OLED. Head-mounted displays can differ in whether they can display just a computer generated image, show live images from the real world or a combination of both. Some head-mounted displays can allow a computer generated image to be superimposed on a real-world view. This is sometimes referred to as augmented reality or mixed reality. Combining real-world view with computer generated image can be done by projecting the computer generated image through a partially reflective mirror and viewing the real world directly. This method is often called “Optical See-Through”. Combining real-world view with computer generated image can also be done electronically by accepting video from a camera and mixing it electronically with computer generated image. This method is often called “Video See-Through”.

In a virtual retinal display, also known as a retinal scan display or retinal projector, a raster display (like a television) is generated directly onto the retina of the eye. The use of a coherent source (such as a laser diode) allows such a system to draw a diffraction limited spot on the retina. The light beam can be intensity modulated to match the intensity of the image being rendered. The user sees what appears to be a conventional display floating in space in front of them. Virtual retinal display system also can show an image in each eye with a very little angle difference for simulating three-dimensional scenes. Another important advantage can be privacy since only the intended user is able to see the image displayed.

Inputs devices can comprise devices such as one or more physical buttons, a touch screen or a portion thereof, a voice recognition device, eye-tracking device, etc. A wide range of haptic devices can also be used and such devices also include motion gestures analysis or interpretation. The devices can be combined with one another (multimodal interaction). For example, a voice command can be confirmed or modulated by an action on a touch sensitive interface.

Touch-sensitive surfaces can comprise sensors to detect intensity of contacts on the touch-sensitive surfaces. Such devices (“force touch”) can use intensity thresholds or ranges of thresholds. Force touch can encode particular user interactions (speed of touch and force can confirm a bolus or even indicate hesitations or particular mood of a patient, since some biometry can be derived from user interactions, as keyboard typing). A touch imparted on the touch-sensitive display can cause a force sensor to undergo an electrical change in resistance that corresponds to a force imparted by the touch. The change in resistance may occur due to a change in geometry of the deflected or displaced material and the change in resistivity of the material arising from micro-changes in the structure of the material under pressure. Generally, between about 1 and 5 N of force may be applied by a user to the touch-sensitive display. Force sensor(s) can be force sensitive resistors, strain gauges, strain sensors, piezoelectric or piezo resistive devices, pressure sensors, or other suitable devices. Various patterns of the force sensors can be used, such as patterns of a single, continuous sensor or patterns of multiple discrete sensors electrically coupled to one another or in isolation. Other patterns, such as multiple force sensor patterns, e.g., bi-directional, multi-grid patterns, may provide increased sensing accuracy with less dependency on the width and orientation of the pattern or the direction of the touch. For example, planar or stacked rosette patterns, such as “tee”, “delta,” and “rectangular” rosettes, may be utilized. Force can refer to force measurements, estimates, and/or calculations, such as pressure, deformation, stress, strain, force density, force-area relationships, thrust, torque, and other effects that include force or related quantities. In some embodiments, the scroll speed or the quantity of data selected (or other logic, with medical significance) can be adjusted in response to the magnitude of force. A gesture can be characterized by, but is not limited to a pinching, sliding, swiping, rotating, flexing, dragging, or tapping motion between or with any other finger or fingers. A single gesture can be performed with one or more hands, by one or more users, or any combination thereof.

Person with diabetes can be visually impaired, since over the years the chronic disease may have caused harm to their eyes and in particular to their retinas. Visual rendering effects, in particular visual magnification, can be triggered as a function of BG value. For example, automatic zoom on priority information can be triggered in case of hypoglycemia. Display of medical information can use one or more visual rendering effects such as magnification, enlargement, zooming, minification, minimization, resizing, masking, scrolling, blinking, morphing, distorting, greyscaling, discretizing and/or coloring. A triggering information can be automatically provided by a sensor, wherein the sensor is selected from a group, or is a combination thereof, comprising: a sensor adapted to perform human face detection, a sensor adapted to evaluate the distance to an human eye, a sensor adapted to detect or analyze breath or smell, a sensor adapted to interpret acceleration data or movements, a sensor adapted to monitor environmental or contextual conditions such as lighting conditions, a sensor adapted do determine an ambient audio level, a sensor adapted to determine a geographic location such as a GPS device, an electroencephalography EEG sensor, an electrocardiography ECG sensor, an electromyography EMG sensor, a sensor adapted do determine a body temperature, a sensor adapted to monitor continuously or discretely a user body parameter such as a blood glucose level or a heartbeat rate or a blood cholesterol level or a blood alcohol level or a blood analyte level. For example, a camera incorporated on the medical device can estimate the mood of the user, or the distance to his face, or estimate the field of vision and provide appropriate responses. In another example, if the accelerometer history indicates that the patient is confused (number of accelerations recorded by the accelerometer above a certain predefined threshold for example), and/or in hypoglycemia state (which may cause the vision to be troubled) the medical system can display some predefined specific data. This automatic mode can be enabled by a cooperation of sensors. Display, user input and data model can be intermingled or combined. Relationships between these three abstractions can be associated with concepts such as influence, feedback, ponderation, limitation, activation, deactivation, control or command. For example, the data priority scheme can influence or drive or control the display logic. For example, if a hypoglycemia probability or event is determined, associated alerts or values can preempt or replace any other display data. User interactivity and machine behavior can be defined by user-defined preferences or by machine learning or driven by rules retrieved from the network.

In some embodiments, the administration of drug is remotely controlled and optionally can be assisted with haptic devices, so that the drug administrator can have realistic feedback of skin penetration or pump manipulation. Haptic components also can be used to train patients or parents (e.g. exercise to prink or perform bolus administration correctly). Advantageously, simulations of bolus delivery can train grandparents which are not constantly trained. In an embodiment, the speed of a touch performed by a user on an interface (for example sensitive to touch) can indicate or determine a parameter of an injection.

In an embodiment, the drug delivery is motorized and the user can define the delivery speed by moving a finger on a touch screen, thus determining the bolus injection which can be performed in real-time, as the gesture is executed. In some embodiments, the drug delivery is postponed in time. In some embodiments, bolus injection profiles are sketched on a touch screen (e.g. with rescaling options).

A diversity of displays can be used. Regarding displays, augmented reality can be used (e.g. a projector or pico-projector can display BG values on the wall or ceiling). Holographic displays can be used. Electronic Braille displays can be used. Touch screens can be used. Display can use force feedback or haptic mechanisms.

Brain machine interfaces can be used. One or more displays can be placed on the glucose sensor and/or on the extension according to the invention and/or on the remote controller of an insulin pump and/or on an associated insulin pump and/or on the smartphone or smart watch associated with the system according to the invention. Displays embedded in smartglasses and/or a smartphone and/or a smart watch can be used. Projectors can be used. A display can comprise one or more of an electronic ink screen or a touch screen or a Braille screen or an OLED screen (or a combination thereof). Opportunistic display can be used (available devices in the vicinity of the system can be accessed and caused to display one or more BG values and/or warnings (in case BG values exceed predefined thresholds). Screenless computing systems also can be used (holograms, virtual retinal display or Retinal Direct, and Synaptic Interface such as Braille or sending signals from electronic devices such as cameras into brains or certain neurons).

A diversity of gestures can be used to enrich the interaction with diabetes management system. Information can scroll. Slide-to-refresh, slide-to-unlock gestures can be used. Force feedback can be used.

For the user experience and the man-machine interaction, a wide diversity of technologies can be used. Speech synthesis can be used (e.g. to enunciate BG values or trends). Text-to-speech can be used (e.g. upon request). Imaging sensors combined with OCR capabilities embedded in software apps can allow the user to acquire an image of the food packaging and automatically extract carbs value for the amount having being eaten. Accelerometers or machine vision can allow to estimate the numbers of swallowing by the patient and to further correlate with carbs intake.

Comprehensibility can be improved for example by using pictograms or audio-guided instead of merely textual information.

Virtual reality and/or augmented reality interfaces can be used to manage diabetes and/or handle the systems according to the invention. User interfaces can be used for the display of information and/or interactivity with the user (e.g. reception of inputs or selections). For example, one or more graphical overlays can be used to indicate the blood glucose value (i.e. graphical elements can be superimposed to the field of view of the user). In an embodiment, the color of the sky can be changed (from dark blue for low values to red for high values; similar or equivalent gradients). In an embodiment, an instant blood glucose value can be opportunistically displayed onto one or more objects in the environment (e.g. windshirm). The display can be effective (i.e. using a projector) or can be virtual (e.g. head-mounted display, with semi-transparent glasses, retinal display, etc.). Display can combine virtual display and real display (for privacy purposes). For example, a red circle can be projected onto a table while the actual value is displayed within said “real” circle. Haptic feedbacks also can be used, for example in combination with displays (for example with progressive intensity). Using virtual and/or augmented reality advantageously can reveal to be non-intrusive and progressively warn the user about instant measures and/or trends (seamless integration, as natural as possible). Via user preferences, notifications can be personalized (for example by defining preferred spatial locations for notifications, depending on types of data, using geofencing rules, etc). For example, a user may prefer notifications to de displayed up in the sky, or down on the floor if walking in the street, etc. Another user may prefer a special part of the body (e.g. the right hand or a specific finger) to be the preferred location place for notifications. Notifications may floating up in the air, be displayed on available hardware screens (picture-in-picture), be centralized with smartphone notifications or to the opposite be separated from it. Snooze and reminders options can be setup with voice commands, gestures, or a combination thereof.

Displays can be in 2D but also in 3D (e.g. stereoscopic), for seamless integration.

Augmented reality and/or virtual reality can be advantageous for diabetes management. For example, an interactive educational diabetes simulator can educate or train patients. Augmented reality (or “mixed reality”) can allow creating a fictional layer on top of the real world context. Said layer can be generated depending on user and/or context data. Virtual media (text, documents, multimedia) can be triggered by location, for example to create a fictional set of events occurring in the real world space. Place-based augmented reality games can be played in specific real-world locations. User experience can be enriched with additional data (text, numerical data, audio, and video). An event in diabetes management, even if properly handled, can be further enriched by using one or more associated simulations, so that the user can learn better and faster. Associated past errors (e.g. insulin stacking) can be reminded to the user and further contextualized. Because a given therapy just occurred, the user can be more receptive to learn further lessons and/or advices. A pet or animal can be simulated for learning purposes (people with prediabetes or children can learn diabetes management in a softly manner). In classrooms, a child with rapidly decreasing blood sugar may be highlighted to the teacher for example (subjective view preserving privacy). A jewel with changing colors worn by the patient can also signal a condition to others (objective view).

Wearable computer can execute software or apps. Wearable computers (e.g. computerized sensors) advantageously can improve diabetes therapy. A diabetes management app for example can display diabetes timecards to the user (e.g. images with CGM readings/trends, insulin on board, meal photos, and other physiologic/activity measures). Users may view (and possibly share) their timecards on-demand or according to configurable notifications. Wearable cameras can capture one or more images of a meal and subtraction methods can estimate the volume of food being eaten (image before and after meal). Meal photos can be snapped using voice commands. Images also can be acquired passively. Sensors (for example in or more teethes) can estimate the quantity of food being ingested (number of mastication movements can be proportional to the food intake). By multiplying the quantity by unitary carbs content, an estimate of total carbs can be determined.

Another use of AR/VR can be to provide users with interactive and “how to” guides (manipulating an insulin pump, an infusion set, etc). For example, the various gestures can be (subjectively) displayed in overlay, step by step, in context so that the user is optimally assisted in the therapy (following the subjective view or a contrario from different angles, at different playback speed, etc)

An AR/VR app can advantageously advise and track dietary choices, i.e. for assisted shopping, upstream before food intake. Healthy choices can be promoted healthy choices in the (physical or virtual) supermarket. Shopping can occur in the reality (e.g. with augmented reality, i.e. with some transparency) and/or in virtual reality (substantially opaque). The abundance of food options in supermarkets, in particular US ones, can make memorizing all of the necessary information cumbersome. A specific app may display caloric density (calories/oz) and/or glycemic index (as well as other type of information useful for diabetes management or other conditions, such as synthetic diet points or other scores). This display can be rendered in audio but also in visual graphics, as the user shops (for example by scanning the barcodes of products loaded into the cart and/or by image recognition and/or by retrieving RFID data, etc). In an embodiment, one or more healthier alternatives can be provided if a poorly scoring food is scanned. Dietary data can be provided in real-time information in a hands-free and private manner. In augmented reality environments, allowed or healthy food can be highlighted and/or unhealthy food can be blurred or otherwise obfuscated (graphical opacity can be configured to detect, track and hide selected food items). Blanked or hided surfaces by content blocking or filtering mechanisms can be replaced by third party content e.g. ads or coupons or other data (e.g. recipes). In virtual reality environments, visual data can be rearranged with more flexibility. Visual density (e.g. quantification of information presented to the user at a given moment, for example counted in number of characters by surface unit and/or in pixels and/or in quantified semiotics) can be configured, so that to provide a good user experience (adaptive cognitive load). Haptic feedback mechanisms can provide seamless information (e.g. unhealthy food can vibrate or be heavier). The rendering of virtual content can occur at any apparent or perceived depth in the virtual space. Implementation of intelligent or optimized depth placement of various elements or instances of virtual content can advantageously prevent clutter in the user's field of view. In some embodiments, the adherence to therapy or attention level of the patient can be optimized or at least preserved (predefined cognitive models can serve as reference). Connections to social media and to peers support can be provided (e.g. providing alerts for calories or food choices, capturing cumulative calorie intake, and health coaching)

Augmented reality and/or mixed reality and/or virtual reality can be enabled by different means. There can be provided an optical viewing device, for example in optical see-through head-mounted display, with an eyeglass-form appearance and a wide see-through field of view. Such particular equipments can allow handling diabetes in such environments. In an embodiment, there is provided a waveguide apparatus which includes a planar waveguide and at least one optical diffraction element (DOE) that provides a plurality of optical paths between an exterior and interior of the planar waveguide. A phase profile of the DOE can combine a linear diffraction grating with a circular lens, to shape a wave front and produce beams with desired focus. Waveguide apparatus may be assembled to create multiple focal planes. The DOE can have a low diffraction efficiency, and planar waveguides can be transparent when viewed normally, allowing passage of light from an ambient environment (e.g., real world). Light can be returned for temporally sequentially passes through the planar waveguide. The one or more optical diffraction elements can be dynamically adjustable. An optical coupler system can couple images to the waveguide apparatus, for example from a projector (e.g. biaxially scanning cantilevered optical fiber tip). In some embodiments, eye tracking mechanisms can be provided. Foveal rendering or foveated imaging (or space variant imaging or gaze contingent imaging) refers to a digital image processing technique in which the image resolution, or amount of detail, varies across the image according to one or more “fixation points.” A fixation point indicates the highest resolution region of the image and corresponds to the center of the eye's retina, the fovea. Optical see-through head-mounted displays can be combined with opaque head-mounted displays (one or more screens arranged in front of the eyes of the user, for example 18 screens paved in a special manner so as to enable ultra-high definition). Transparency can be adjustable. Various devices can be used to display augmented and/or virtual viewpoints (visual accommodation via magnifying optics, mirrors, contact lenses, or light structuring elements), non-see-through displays of light emitting elements (LCDs, OLEDs, vertical-cavity-surface-emitting lasers, steered laser beams, etc.), see-through displays that simultaneously allow users to see the real world and artificially generated images (for example, light-guide optical elements, transparent and polarized OLEDs shining into close-focus contact lenses, steered laser beams, etc), contact lenses with light-emitting elements (also combined with specialized complimentary eyeglasses components), implantable devices with light-emitting elements, and implantable devices to stimulate the optical receptors of the human brain.

AR/VR devices can optionally include one or more haptic devices or components, operable to provide a tactile sensation to a user. For example, a haptic device can provide a tactile sensation of pressure and/or texture when touching virtual content (e.g., virtual objects, virtual tools, other virtual constructs). The tactile sensation can replicate a feel of a physical object which a virtual object represents. In some embodiments, haptic devices can be worn by the user (user wearable glove, haptic totems, etc). One or more devices can detect and interpret user gestures into commands. Some gestures can be discretely performed while some others can be demonstrative (e.g. intention to capture images and/or audio of other persons). Some gestures may also be culturally acceptable (some gestures may be considered offensive in some cultures and should be avoided).

The medical system according the invention can comprise a brain-computer interface (BCI) or mind-machine interface (MMI) or direct neural interface (DNI) or brain-machine interface (BMI). Such expressions designate a direct communication pathway between an enhanced or wired brain and an external device. A brain-computer interface encompasses any form of controlling a computer via a direct electrical connection to the human body. The patient can “feel” the blood glucose level, continuously or on demand, and for example can trigger or otherwise control the delivery of insulin (or other drugs). The user also can control the various user interfaces described herein (in particular any one of the AR/VR embodiments). BCIs can be invasive or not, EEG based or non EEG-based (e.g. pupil-size oscillation). BCIs generally use a combination of EEG (electroencephalography), EMG (electromyography), EKG (electrocardiography), and accelerometer data. BCIs and eye-tracking can be combined.

In some embodiments, for example involving a patch-pump or a micro pump which are not provided with screens, display can be deported. Display devices can be integrated in smartphone but also in head-mounted displays. A head-mounted display is a display device, worn on the head, which has a small display optic in front of one (monocular) or each eye (binocular). A typical head-mounted display has either one or two small displays with lenses and semi-transparent mirrors embedded in a helmet, eye-glasses (also known as data glasses) or visor. The display units are miniaturized and may include CRT, LCDs, or OLED. Head-mounted displays differ in whether they can display just a computer generated image, show live images from the real world or a combination of both. Some head-mounted displays allow a computer generated image to be superimposed on a real-world view. This is sometimes referred to as augmented reality or mixed reality. Combining real-world view with computer generated image can be done by projecting the computer generated image through a partially reflective mirror and viewing the real world directly. This method is often called “Optical See-Through”. Combining real-world view with computer generated image can also be done electronically by accepting video from a camera and mixing it electronically with computer generated image (“Video See-Through”). In such devices, the attention of the user shall be properly managed to avoid unnecessary distractions. Appropriate areas in the field of vision have to be determined. The balance and compromises to be made correspond to the present invention which mechanisms allow for a balanced compromise, ponderation or selection of data to be displayed (with respect to substance), and the visual effect such as placement, surface, area, still or animated modes (with respect to the form).

In some embodiments, retinal display is used (with a laser, monochromic if not colored images can be obtained by direct or indirect projection onto the retina). In some embodiments, the user can be wearing a virtual retinal display, also known as a retinal scan display or retinal projector. Such a display technology draws a raster display (like a television) directly onto the retina of the eye. The use of a coherent source (such as a laser diode) allows the system to draw a diffraction limited spot on the retina. The light beam is intensity modulated to match the intensity of the image being rendered. The user sees what appears to be a conventional display floating in space in front of them. Virtual retinal display system also can show an image in each eye with a very little angle difference for simulating three-dimensional scenes. Another important advantage is privacy since only the intended user is able to see the image displayed.

Display devices can cooperate (display can be distributed). One main screen or display may handle the display of all or part of the medical data, but several displays may handle in cooperation the “global” display (i.e. the interaction towards the user). For example, a glucometer may display some type of information (such as blood glucose and basal information), while the pump would “specialize” in maintenance information. A CGM based device (continuous monitoring device) can only display blood glucose and probabilistic expected evolution of the glucose level. When the blood glucose is decreasing too rapidly, this acts as the “triggering information”. Either the CGM can magnify or highlight the current measurement either it can send a command for any type of rendering effect to the central display implemented on the pump and/or on the remote controller and/or glucometer. Prompts can be remotely commanded (parents of the child with the chronic disease may be prompted by an active window surging on their desktop, because of a triggering information such as a fast decrease in blood glucose

User interactivity and machine behavior can be defined by user-defined preferences or by machine learning or driven by rules retrieved from the network. The assessed state of a user or patient can indeed drive the interactivity model. A user profile can comprise data such as the age of the patient, user preferences (in terms of display, reminders, alerts, type and frequency of desired interaction), habits (typical agenda and schedules, date of anniversaries of family members, . . . ) health statistics, personal rules, as well as sources of data in which to retrieve—in real-time or not—additional personal data (such as email or social network website account for example). For example, just taking into account the age of the patient can lead to an effective user interaction. Above 60 years old, the system may introduce a bias in the pump preferences to increase the probability of switching to a zoom mode when certain criteria are met (automatic triggering information). These settings can be made manually (the user editing his permanent preferences) or can be set up automatically. Said display preferences also can comprise particular rules. For example, when the presence of certain persons are detected in the vicinity of the patient wearing the medical device, a particular display mode can be deactivated or switched off when handled by the doctor and no longer by the child. User preferences also can be edited. For example, a user can edit his own list of priority ranks, each information type being associated with a priority rank (bolus dose can be associated with rank 1, previous bolus the day before is associated with rank 2, date and time is associated with rank 3 for example). In some embodiments, logic rules governing and possibly distorting situation awareness can be deactivated on demand (raw data can be accessed with no data filters, while refined and sophisticated data also can be accessed on demand).

While “proactive” user interaction is possible, a return to the normal state and behavior of a medical device can remains possible. When triggered, a user interface can return into its “passive” state. An alternative consists in displaying, as a “second chance” mode, a second subset of data to the user (or according to an alternative manner). Successive user commands can enable such “switches” (for example one first press on a button results in a first display mode, a second press results in another mode, and at the third press the system gives up and returns to its initial state). In this view, some opportunities are provided to the machine to show its “intelligence”, but after a (limited) number of trials, the machine can return in passive or obeisance mode.

### Association Schemes

In some embodiments, parts of the medical system according to the invention can be arranged and/or configured according to association schemes.

Subparts of the medical system can be (e.g. physically) arranged and/or (e.g. logically) configured (or adapted) according to different schemes. To get a robust combination, one or more components can be redundant (duplicated or triplicated). The components of the system can be distributed (e.g. “body area network”) and/or centralized. The association between the one and more sensors and/or the one or more sensors and/or the one or more drug delivery actuators can be performed in different ways. Association can reversible (e.g. releasable) or irreversible. Association can one or more of adhesive e.g. Gecko-based adhesive, aerogel, glue, Velcro, magnetic (releasable), electrical, pressure-based, etc. The one and more sensors and/or the one or more sensors and/or the one or more drug delivery actuators can be located adjacent from another, or at remote distance (body area network, cloud, etc).

Association or pairing between apps can use QR codes, or barcodes, or tokens, or communication protocols, with or without the intervention of a user. The disconnection of one or more parts can trigger an alarm.

The extension can be integrated or inserted or melted within an e-textile, or use flexible electronics. The extension can be 3D printed. It can be integrated in textile.

Regarding hardware, flexible wired connections can be used. FPGA circuits can be used to provide faster responses times and higher resistance to cyber-attacks. The architecture of the system can be fractal. Cloud computing resources can be used (or “grid”).

### Communication Schemes

In some embodiments, the medical system or parts thereof are arranged and/or configured according to one or more communication schemes.

Various communications means (e.g. Wifi, Bluetooth, etc), protocols, modulations (e.g. CDMA), medium/media (e.g. wired/wireless) or data transport schemes can be used.

Communications can use a plurality of networks, comprising NFC, ibeacon, Wi-Fi, Li-Fi, Wimax, 2G, 3G, 4G and 5G.

The different devices and/or sensors can use a diversity of communications schemes and/or networks topology (e.g. peer-to-peer, mesh, ad hoc, centralized, etc) and/or technology (Bluetooth Low Energy BLE, Wifi, Li-Fi, ibeacon, etc)

In some embodiments, the system can be part of a mesh or ad hoc network (loosely coupled devices, offering ephemeral controllability of the global system).

Data communication can use fiber optics and/or lasers. In an embodiment, quantum key distribution can be used for the different parts of the architecture to define and share one or more secret keys, the further classical encryption of further data exchanges using said keys. In an embodiment, post-quantum cryptography can be used.

Software-defined radio can be used.

In order to avoid interception or eavesdropping, medical data can be streamed (i.e. no complete data can be captured at a given moment), at least in parts.

Cognitive radio technology, also known as smart radio can allow different radio technologies to share the same spectrum efficiently by adaptively finding unused spectrum and adapting the transmission scheme to the requirements of the technologies currently sharing the spectrum. This dynamic radio resource management is achieved in a distributed fashion and relies on software-defined radio.

A cognitive radio (CR) is an intelligent radio that can be programmed and configured dynamically. Its transceiver is designed to use the best wireless channels in its vicinity. Such a radio automatically detects available channels in wireless spectrum, then accordingly changes its transmission or reception parameters to allow more concurrent wireless communications in a given spectrum band at one location. This process is a form of dynamic spectrum management.

Li-Fi technology can be used. Li-Fi can facilitate high-speed data transmission via pulsating light sources.

Communications can use any of a plurality of communications standards, protocols and technologies, including but not limited to Global System for Mobile Communications (GSM), Enhanced Data GSM Environment (EDGE), high-speed downlink packet access (HSDPA), high-speed uplink packet access (HSUPA), Evolution, Data-Only (EV-DO), HSPA, HSPA+, Dual-Cell HSPA (DC-HSPDA), long term evolution (LTE), near field communication (NFC), wideband code division multiple access (W-CDMA), code division multiple access (CDMA), time division multiple access (TDMA), Bluetooth, Wireless Fidelity (Wi-Fi) (e.g., IEEE 802.11a, IEEE 802.11ac, IEEE 802.11ax, IEEE 802.11b, IEEE 802.11g and/or IEEE 802.11n), voice over Internet Protocol (VoIP), Wi-MAX, a protocol for e-mail (e.g., Internet message access protocol (IMAP) and/or post office protocol (POP)), instant messaging (e.g., extensible messaging and presence protocol (XMPP), Session Initiation Protocol for Instant Messaging and Presence Leveraging Extensions (SIMPLE), Instant Messaging and Presence Service (IMPS)), and/or Short Message Service (SMS).

### Security Schemes

In some embodiments, the medical system or parts thereof are arranged and/or configured according to one or more security schemes.

Security schemes can comprise a Physically Unclonable Function and/or a challenge-response test and/or a True Random Number Generator.

Various mechanisms can be used to improve security and/or safety of drug delivery according to the invention.

Communications can be encrypted and/or obfuscated. Security of the system according to the invention or of specific part thereof can be protected using one or more of the technologies or mechanisms comprising asymmetrical encryption like AES, public key encryption like PGP or GPG, physically unclonable function or PUF, cryptoledger or blockchain, proofs-of-work, quantum key distribution, post-quantum cryptography, etc. Also steganography can be used (e.g. diabetes reports can be concealed a file, message, image, video or within another file, possibly of no particular subjective interest).

Biometrics can be used to grant access to the system. End-to-End encryption can be used. Token-Based Access Control can be used.

Cyber attacks against the system can be prevented by the use of Turing tests (e.g. challenge-response for bolus administration after a value is buffered but not actually injected, etc). In an embodiment, the extension according to the invention can serve as a gateway for security purposes. For example, the extension can implement or participate to a Turing challenge (e.g. a CAPTCHA), ensuring that a human being is forming request to retrieve a BG value (beyond the injection of insulin which can benefit from such a testing scheme). The extension also can embed one or more ciphering keys, which can be required for a global chain of devices to properly work (Digital Rights Management) or to be authorized to function. Security schemes can advantageously be used to impede man-in-the-middle attacks (a fake NFC reader can request BG values, for example to further falsify or attack an artificial pancreas embodiment). In an embodiment, the extension can continuously map available devices for diabetes management in the vicinity and handle encryption keys accordingly, with genuine and/or authorized devices for the management of diabetes. Regarding data communication, onion routing can be used.

“Secure boot” or “verified boot” can be used. One or more described subsystems according to the invention can use secure and/or verified boot. One or more devices of the diabetes management system can be secured, by a “secured boot” or a “verified boot” (for example, hash values at startup can be compared with authorized values). Such embodiments advantageously can defeat sabotage or cyber attacks. If not successfully verified, a safety-critical device can be executed in a downgraded state (e.g. specific functions can be forbidden for execution)

Hard switch or hard-off switch can be used (for example to deactivate enhanced mode of diabetes management, or particular rules, which may reveal to dysfunction).

Wired communication can be required to avoid eavesdropping or attacks (such as man-in-the-middle attacks). While wireless communications are generally efficient, it may reveal advantageous to require wired connections, in particular for bolus injections. Wireless communications indeed can be attacked or eavesdropped, while wired connections between subsystems of diabetes management would impose a physical intervention which would then be easily detected (i.e. prevented, the user monitoring physical integrity of the system).

Onion-routing or TOR networks can be used. Onion-routing can be combined by techniques for preserving anonymity (e.g. proxies, Chaum mix networks, P3P, etc).

Communication of medical data can use the bitorrent protocol, for example combined with TOR and/or zero-knowledge mechanisms.

Some hardware can be triplicated. Triple modular redundancy (TMR) is a fault-tolerant form of N-modular redundancy, in which three systems perform a process and that result is processed by a majority-voting system to produce a single output. If any one of the three systems fails, the other two systems can correct and mask the fault. TMR can be used for different parts of the invention. Triple modular redundancy hardware can be faster than Hamming error correction software. In diabetes management most critical and/or weak and/or fragile hardware and/or software parts thus can be robustified.

Computer security of hardware and/or software embodiments can be improved using mechanisms comprising formal verification of code (e.g. automated theorem proving), two-factor authentication, regular security patches and updates, use of a security scanner, automated audit trails, dongles, trusted platform modules, intrusion-aware cases, drive locks, disabled USB ports, use Virtual Private Networks (VPNs), computer case intrusion detection (e.g. push-button switch), encrypt hard drives, biometric validation (such as thumb print readers), use of secure coding techniques, access control lists, interference shields, etc. Trusted computing techniques can be used (e.g. using one or more of an endorsement key, secure input and output, memory curtaining/protected execution, sealed storage, remote attestation, etc).

In some embodiments, one or more parts of the invention (e.g. a sensor and/or an injection device such as an insulin pump) can be optionally secured with a physically unclonable function (PUF). A PUF is a physical structure which is generally easy to evaluate but hard to predict. An individual PUF device is generally impossible to duplicate, even given the exact manufacturing process that produced it. In this respect it is the hardware analog of a one-way function (e.g. a challenge-response). A PUF can be used for key generation (enabling authentication for example). A PUF can provide a collection of responses with predefined ranges of values and properties (randomness, aging, entropy, etc). Using one or more PUFs in medical devices is advantageous.

In some critical embodiments, Quantum Key Distribution (QKD) can be used. Quantum key distribution can be enabled on mobile devices. QKD is used to produce and distribute a key, not to transmit any message data. The key can then be used with any encryption algorithm, such as AES. In some embodiments, a pseudo random number generator can be used. In some embodiments, a quantum random number generator can be used.

In some embodiments, the medical system according to the invention, or parts thereof, can be arranged and/or configured according to one or more cryptographic schemes.

Cryptographic schemes comprise a Quantum Key Distribution mechanism and/or post-quantum cryptography and/or quantum-safe cryptography and/or crypto-ledger and/or one or more smart contracts configured to control or influence operations of the medical device and/or communications thereof. Public keys schemes and/or symmetrical encryption can be implemented.

Security of networks of sensors (e.g. Internet of Things) can be performed in several manners, for example by using one or more symmetric keys with gateways, by selectively protecting vital and immutable packet parts with message authentication code(s) with encryption, or by using message authentication codes. Other mechanisms include one or more of puzzle-based defense mechanisms, ad-hoc security domains, chains of certificates, privacy aware identifiers used to prevent unauthorized user tracking, built-in mobility signaling or combinations thereof.

### Medical Management Rules

In some embodiments, the medical system, parts thereof and/or the control thereof can be arranged and/or configured according to one or more medical management rules.

Medical management rules can be specific to/for particular medical conditions, for example for diabetes. Some rules can be FDA-regulated. Some others may not be (private use).

Medical data can comprise blood glucose data, bolus dose, bolus type, basal rate, temporary basal rate, calibration reminder, occlusion probability or event, leakage probability or event, hypoglycemia probability or event, hyperglycemia probability or event, ketosis or ketoacidosis probability or event, maintenance event or reminder such as cartridge or reservoir replacement or battery replacement.

Advantageously, the implementation of medical rules can place the patient at the heart of diabetes management (personalized system), can provide algorithms as “tools”, among other “tools” (place them as concurrent “offers”), can set transparency at all levels (hardware specifications, Open Source Software, algorithms assumptions and models). Open or free software can lead to faster development, to a kind of “immortal” code (i.e. fork-able code base).

In some embodiments, the metabolism of the patient can be measured and/or estimated and/or simulated and/or computed, directly and/or indirectly, statically and/or dynamically.

Diabetes management rules can use one or more metrics. Anonymized data can be aggregated and sensors analytics can be public. Botnets (collection of computers) can data-mine the aggregated data. The rules can be configurable in the Cloud by the patient. In some embodiments, the patient can configure time intervals and/or thresholds for notifications.

A rule can be for example “if the heart rate is superior to 140 beats per minute and more than 75% of prediction algorithms with a 5% error threshold determine the advent of a hypoglycemia within the next 15 minutes then execute audio alarm, both local and distant”. Another example of a rule can be “deactivate the preceding rule if a pizza has been declared eaten less than 4 hours ago”. Rules can handle the handling of exceptions by general amplifiers or attenuators, e.g. “increase all thresholds applicable to basal insulin by 20% if corporal temperature has exceeded more than 40° C. during 2 hours over the last past 12 hours” or “increase basal insulin by 20% if accelerometer date indicate a sport exercise increased by 10% compared to normal average situation” or “if patient moves more than threshold N, apply rule number M”. Rules can handle specific triggers e.g. “trigger measurements every 5 mn in 3 hours and if BG value at 10 pm exceeds 220”. Rules can handle reminders e.g. “alarm on parent's smartphone in 4 hours unless BG value is above 130”.

In an embodiment, rules are expressed in natural language by the patient and/or parent and further converted into formal logical rules. In an embodiment, fuzzy logic is used.

Heuristics (machine-readable) and/or rules (human-readable) can be implemented on request in the system according to the invention.

Rules can be ordered hierarchically.

In an embodiment, a plurality of logical or software rules can govern the hardware according to the invention. The personalization or configuration of diabetic rules and the further assembly or combination of such rules can lead to a DIY Do-it-Yourself system. In an embodiment, each rule can be associated with a FDA score, each combination can be associated with a specific score (e.g. in terms of reliability, performance, systemic risks assessments, etc). Particular combinations may be forbidden. Some other rules may be recommended. Rules or combinations of rules (“packages”) can be downloaded and further installed. Rules can be protected by DRM. Some can be open-sourced, while some others can remain in binary forms. Some can be insured, some others not.

The correlations or covariance or invariance or coupling of sensors (by pairs or more) can be determined, in many different independent or combined ways. Software agents can crawl the web corpus to establish correlations and patterns, the identification of critical parameters, specific to an individual. Human crowd sourcing also can browse and back-test data to identify composite data combinations improving hypo detections. Social networks can be used (e.g. estimation of carbohydrates values of images of meals).

The physiology of the patient can be modeled, for example with deep learning. A virtual clone of the patient can enable to test injections and estimate future BG values or trends.

The User Interface to define or use or configure rules can use gamification. Head-mounted displays can be used (or “glasses” or virtual reality helmets). Haptic interfaces can allow the patient to handle, visualize and configure personalized rules for diabetes management.

The rules can be configured in an interactive system. One or more intermediaries can handle, filter and enhance the data at each step of the algorithmic chain.

A rule can be location-based. A rule can be locked, conditionally authorized (depending on the context, requiring payment, etc). A rule can be free, require payment or can be installed for free with advanced features and/or settings requiring a payment.

Machine-to-machine communications can occur, for example between modeled physiologies (set of rules 1 made for patient profile 1 can be tested with a profile 2).

The rules for diabetes management can be personalized. Personalized rules can be scored by comparison with FDA approved diabetes management rules. Approved diabetes management rules can take into account systemic risks (i.e. specific combinations of sensing and delivery devices which could not allow patients to take appropriate measures).

Diabetes management rules (“rules”) can be use priority mechanisms (a rule can be associated with a priority and a plurality of competing rules can be executed in parallel, a selection and/or coordination among rules results or predictions can be performed). Diabetes management rules can preserve the patient's privacy. Rules can be public (standardized rules, with no configuration data for example) or private (specifics can be confidential, for example the amount of boluses, so that to avoid excessive surveillance attempts by insurance companies for example). Diabetes management rules can be probabilistic. Diabetes management rules can be programmable, in part or in full. Diabetes management rules can be advertized and/or ranked. Via social networks, patients can rate, score or comment one or more rules or recommendations so that to improve learning curves and/or suggest rules' improvements (for example). Diabetes management rules can be simulated (installed in a sandbox, to estimate resulting BG values knowing the lifestyle and past BG values of a patient). Search engines can index and rank by relevancy available diabetes management rules according to the user profile. Diabetes management rules can include structured testing, reminders or alarms, bolus or basal injection patterns or complex rules based on sensor's data (heart rate, audio level, wetness, vibrations etc).

Diabetes management rules can be scripted. In procedural knowledge, scripts are like frames, except the values that fill the slots must be ordered. A script can be a structured representation describing a stereotyped sequence of events in a particular context. Scripts can be used in natural language understanding systems to organize a knowledge base in terms of the situations and conceptual transitions that the system should understand.

Diabetes management rules can comprise or use smart contracts. Smart contracts comprise computer protocols which facilitate, verify, or enforce the negotiation or performance of a contract (or that make a contractual clause unnecessary). Contractual clauses can be made partially or fully self-executing and/or self-enforcing, reducing transaction costs associated with contracting. The provision of tangible devices and/or software rules can be regulated with the use of such smart contracts. In an embodiment, physical objects can be micro-tagged with contractual requirements (e.g. payment can be conditional or enforced for certain types of uses of certain predefined types of information).

BG values can be presented to the user by “pull” and/or “push”. The user can request BG values (“pull”) and/or BG values can be presented to the user (“push”). The monitoring of audio (for example combined with agenda data) can allow to present BG values at appropriate or optimized time-frames.

In an embodiment, users can subscribe to one or more “channels” (e.g. trustable persons or corporation entities) delivering or proposing diabetes management rules.

The social graph of users of diabetes management rules can be analyzed. Super-nodes can be identified (e.g. users with intense social activity, trusted users, influencers, etc).

Other aspects are now described. According to the invention, software architecture can comprise an abstraction of the run-time elements of a software system during some phase of its operation. A system can be composed of one or more plurality of levels of abstraction and one or more phases of operation, each with its own software architecture. A software architecture can be defined by a configuration of architectural elements—components, connectors, and data—constrained in their relationships in order to achieve a desired set of architectural properties. A component can be an abstract unit of software instructions and internal state that provides a transformation of data via its interface. A connector can be an abstract mechanism that mediates communication, coordination, or cooperation among components. A datum can be an element of information that is transferred from a component, or received by a component, via a connector. A configuration can designate the structure of architectural relationships among components, connectors, and data during a period of system run-time. Data-flow properties can comprise efficiency, scalability, simplicity, evolvability, extensibility, customizability, reusability, visibility, portability and reliability.

“Diabetes management” according to the invention for example designate the evaluation of carbs of a meal given one or more pictures thereof, determination of bolus and/or basal value, analysis of trends and predictions based on raw data, or more generally any therapeutic measure, determination, action or evaluation.

Diabetes management according to the invention can use involve various data sources, e.g. human mechanisms and/or machine technologies. For example, diabetes management can use one or more of data mining, deep learning, beam search, LDPC-codes, neural network, etc.

Diabetes management according to the invention can use “machine learning”, e.g. supervised learning, for example by identifying of patterns in BG values (e.g. postprandial profiles, exercise profiles, at night, etc).

Diabetes management according to the invention can use deep-learning (this field for example comprises one or more of techniques comprising sparse coding, compressed sensing, connectionism, reservoir computing, liquid state machine, echo state network, supervised learning, classification, regression, clustering, dimensionality reduction, structured prediction, anomaly detection, neural nets, machine learning venues, artificial neural networks, deep neural network architectures, back propagation, convolutional neural networks, neural history compressor, recursive neural networks, long short term memory, deep belief networks, convolutional deep belief networks, deep Boltzmann machines, stacked (de-noising) auto-encoders, deep stacking networks, tensor deep stacking networks, spike-and-slab RBMs, compound hierarchical-deep models, deep coding networks, deep q-networks, networks with separate memory structures, LSTM-related differentiable memory structures, semantic hashing, neural Turing machines, memory networks, pointer networks, encoder-decoder networks, multilayer kernel machine, etc).

Diabetes management according to the invention can use web services. A Web service is a service offered by an electronic device to another electronic device (machine-to-machine communication), communicating with each other for example via the World Wide Web. Major classes of Web services comprise REST-compliant Web services (manipulation of representations of Web resources using a uniform set of stateless operations) and Arbitrary Web services (in which the service may expose an arbitrary set of operations). In particular, a Web API is a development in Web services with a simpler representational state transfer (REST) based communications. RESTful APIs do not require XML-based Web service protocols (SOAP and WSDL) to support their interfaces.

Diabetes management according to the invention can use web services service-oriented architecture (SOA). SOA is an architectural pattern in computer software design in which application components provide services to other components via a communications protocol, typically over a network. SOA generally encapsulates application logic in services with a uniformly defined interface and makes these publicly available via discovery mechanisms.

Diabetes management according to the invention can use so-called web 2.0, mashups of applications or APIs. Web 2.0 designates the ability of visitors to contribute information for collaboration and sharing. Web 2.0 applications generally use RESTful web APIs and AJAX based user interfaces, utilizing web syndication, blogs, and wikis. Diabetes management according to the invention can use service-oriented business applications (SOBAs).

Diabetes management according to the invention can use technologies of the “Internet of Services”, wherein people, machines, and goods have access via the network infrastructure. Micro services can be used (interpretation of service-oriented architectures used to build distributed software systems, by using technology agnostic protocols).

Diabetes management according to the invention, for example diabetes management rules, can use various time mechanisms e.g. time-to-live (TTL), timers, specific diabetes/biological time, etc.

Diabetes management according to the invention can use Error-correction code ECC or Forward error correction FCC (this field for example refers to or comprises one or more of techniques comprising concatenated FEC codes for improved performance, low-density parity-check LDPC, turbo codes, etc).

Diabetes management according to the invention can use turbo codes or turbo codes (one or more of AN codes, BCH code, Berger code, Constant-weight code, Convolutional code, Expander codes, Group codes, Golay code, Goppa code, Hadamard code, Hagelbarger code, Hamming code, Latin square based code for non-white noise, Lexicographic code, Long code, Low-density parity-check code, also known as Gallager code, LT code, Fountain code, online code, raptor code, reed-Solomon error correction, reed-Muller code, repeat-accumulate code, repetition codes such as Triple modular redundancy Spinal code, Tornado code, Walsh-Hadamard code, Viterbi algorithm, Soft-decision decoding, Interleaver BCJR algorithm, serial concatenated convolutional codes, turbo equalizer

Diabetes management according to the invention can use fuzzy-logic (natural language interfaces, e.g. rules expressed in a way which is easy to understand and/or modify by the user and which is manipulatable by the computer). This field for example refers to or comprises one or more of techniques comprising adaptive neuro fuzzy inference system ANFIS, artificial neural network, defuzzification, expert system, false dilemma, fuzzy architectural spatial analysis, fuzzy classification, fuzzy concept, fuzzy Control Language, fuzzy control system, fuzzy electronics, fuzzy subalgebra, fuzzyCLIPS, High Performance Fuzzy Computing, IEEE Transactions on Fuzzy Systems, Interval finite element, Neuro-fuzzy techniques, noise-based logic, rough set, sorites paradox, type-2 fuzzy sets and systems, vector logic)

Diabetes management according to the invention can use Bayesian inference (this field for example refers to or comprises one or more of techniques comprising admissible decision rule, Bayesian efficiency, Bayesian probability, Probability interpretations, Bayes' theorem, Bayes' rule, Bayes factor, Bayesian network, Prior Posterior, Likelihood Conjugate, prior, Posterior, predictive, Hyperparameter, Hyperprior, Principle of indifference, Principle of maximum entropy, Empirical Bayes method, Cromwell's rule, Bernstein-von Mises theorem, Bayesian information criterion, Credible interval, Maximum a posteriori estimation, Bayesian linear regression, Bayesian estimator, Approximate Bayesian computation, Bayesian hierarchical modeling, Bayesian Structural Time Series, Monty Hall problem

Diabetes management according to the invention can use LDPC-codes (this field for example refers to or comprises one or more of techniques comprising belief propagation, graph theory, Hamming code, linear code, sparse graph code, expander code).

Diabetes management according to the invention can use other capacity-approaching codes (e.g. comprising serial concatenated convolutional codes, online codes, fountain codes, raptor codes, repeat-accumulate codes, Tornado codes or Polar codes).

Diabetes management according to the invention can comprise one or more diabetes management rules. Algorithms associated with diabetes management rules can be executed locally, i.e. on a computing device in the vicinity of the user. Alternatively or as a complement (elastic computing), remote computing resources can be used.

Privacy-techniques can be used. A range of techniques can be combined with embodiments of the invention. Various techniques can be used, possibly in combination. Some of these techniques or steps are described hereinafter.

“Homomorphic encryption” can be used. Homomorphic encryption is a form of encryption that allows computations to be carried out on ciphertext, thus generating an encrypted result which, when decrypted, matches the result of operations performed on the plaintext. Such use advantageously enables to preserve privacy.

“Secure multi party computation” can be used. SMPC is a subfield of cryptography enabling the parties to jointly compute a function over their inputs while keeping those inputs private

“Virtual Party Protocol” can use virtual parties and mathematics to hide the identity of the parties.

“Secure sum protocols” can be used to allow multiple cooperating parties to compute sum function of their individual data without revealing the data to one another

“Differential privacy” can be used. DP is a technique for releasing statistical information about a database without revealing information about its individual entries. DP can maximize the accuracy of queries from statistical databases while minimizing the chances of identifying its records.

“Quasi-identifiers” can be used. When combined, QI become personally identifying information.

“Exponential Mechanisms” can be used. With EM, one can output a synthetic dataset in a differentially private manner and can use the dataset to answer queries with good accuracy.”

“K-anonymity” can be used. Given person-specific field-structured data, produce a release of the data with scientific guarantees that the individuals who are the subjects of the data cannot be re-identified while the data remain practically useful.

Diabetes management and/or algorithms can use can comprise interpolation steps, iterative, recursive steps.

Diabetes management can use feed-forward mechanisms (with or without feedback mechanism). These mechanisms can relate to control theory, physiology/biology or computing and can prove advantageous for diabetes management. Feed-forward designates an element or pathway within a control system which passes a controlling signal from its external environment to a load elsewhere in its external environment. A feed-forward system responds to its control signal in a pre-defined way without responding to how the load reacts. In contrast, a system with a feedback mechanism adjusts the output to take account of how it affects the load (the load itself can vary unpredictably, and the load is generally considered to belong to the external environment of the system). In a feed-forward system, the control variable adjustment is not error-based: it is based on knowledge (e.g. in the form of a mathematical model) of the process and knowledge about or measurements of the process disturbances. Pure feed-forward control without feedback can be called “ballistic”, because once a control signal has been sent, it cannot be further adjusted (any corrective adjustment must be by way of a new control signal). By contrast, “cruise control” adjusts the output in response to the load that it encounters, by a feedback mechanism.

Algorithms for diabetes management (e.g. evaluation of carbs of a meal given a picture thereof, determination of bolus value, etc) can involve various sources and/or technologies, comprising crowd sourcing and social networks, or human evaluation (e.g. by doctors) along with machine algorithms. Advices (e.g. proposals of rules and/or values) can be taken into account, by “pull” (e.g. upon request by the patient, for example in an uncertain situation or in a hurry) and/or “push” (for example, different opinions are collected and ranked, for later display to the patient). Some examples are further described. In a first example, real-time blood or interstitial glucose values are published on the internet possibly anonymized. One or more (qualified) doctors then can trigger alerts or provide general purpose advices. Social networks, i.e. one or more persons following the individual can also contribute (e.g. monitor dangerous trends, call by phone if thresholds are crossed). In a second example, the patient can take one picture or image before the meal and another after the meal. Said images can be uploaded on the internet and published, for example in a social network. Both humans and machines can concurrently tentatively estimate the carbs intake. By subtracting images, machine vision can determine or estimate the volume of carbs having being ingested, and by reference to volumic average carbs values then determine a total amount of carbs. Human followers also can try to estimate carbs value. Even after the initial picture of food is uploaded and published, humans and machine can start evaluations.

### Social Mechanisms

In some embodiments, the medical system, parts thereof and/or the control thereof can be arranged and/or configured according to one or more social mechanisms.

A diversity of social features can be used. Crowd of users can evaluate meal carbs, discuss BG values and/or trends, and comment on tips and tricks to handle diabetes. Real-time encrypted chats among peers can encourage dialog and improve therapy or adherence to therapy. Users can take pics of meals and cooperatively evaluate carbs contents, along machine vision or recognition.

In some embodiments, standards can be used (facilitating interoperability, hence faster and wider adoption). For particular aspects, at least temporarily, proprietary technologies can be used to optimize user “lock-in” (in turn facilitating return on investment and further development). Open standards can be used. Likewise, APIs can be used (or open APIs).

Usage data can be gathered and anonymized. Statistics can be derived from this collection. Homomorphic encryption can be used (logical operations performed on encrypted data).

Some systems according to the invention can be operated in so-called “stealth” mode or “camouflaged” mode. For example, diabetes management app can be disguised into a classical software app and the reference to diabetes can be obfuscated. Likewise, the injection of insulin can be branded or shown as the injection of vitamins or other less socially-intriguing substances. Insulin pens can be camouflaged into ink pens or other gadgets. Insulin pumps can be camouflaged into GPS devices or mobile devices (mobile phone), embedded into a teddy bear, etc. Infusion sets can be camouflaged with tattoos (temporary or permanent). Tubing can be camouflaged into old school telephone cords or other power cords, if not jewelry.

### Enemy Management Schemes

In some embodiments, the medical system, parts thereof and/or the control thereof can be arranged and/or configured according to one or more energy management schemes.

A diversity of energy sources can be used. For example, the battery powering the insulin pump can be disposable or rechargeable. Renewable energy can be used. The source of energy can include photovoltaic energy.

Battery can use different technologies and combinations thereof: lithium-ion, lithium-iron and lithium-sulfur for example.

The source of power can use rechargeable battery or a dynamo or a gravity source of energy. A fuel cell can be used.

Energy management can use various mechanisms, including light or deep hibernations, screensavers, etc. Electronic circuits selectively can be powered-off (for example according to criticity levels associated with the different electronic circuits constituting the medical system). Various cooling off systems can be used.

### Time and/or Space Schemes

In some embodiments, the medical system, parts thereof and/or the control thereof are arranged and/or configured according to one or more time and/or space schemes.

Dimensions of sensors and/or actuators can be different (e.g. macro or micro-scales).

Structured testing can comprise different schedules to retrieve data.

Measurements can be performed “continuously” and/or “continually” and/or ““intermittently” (regularly or irregularly) performed.

BG measures can be regular or irregular, periodic or a-periodic, intermittent, opportunistic (triggered by predefined event, available bandwidth, etc).

BG values can be logged. History of logs can be archived. Logs can be encrypted.

The frequency of sampling can be event-driven (e.g. movement while sleeping)

The sensor can determine the presence of one or more biomarkers.

In an embodiment, the glucometer is implanted under the skin while NCF or equivalent communications enable the retrieval and/or injection of data.

Time management is an important factor in diabetes management. Various time management can be implemented, for example using one or more of a timeout, a timer, a timestamp. Time can be divided in hours minutes and seconds but specific diabetes time units can be defined, for example in relation with residual insulin. Graphical indicators can be implemented (remaining time, residual insulin, time before hypoglycemia, etc). Specific custom clock faces can be determined for diabetes. Adherence to therapy can be encouraged by adapted user interfaces (e.g. indicating progress, marking rewards, providing warnings, etc).

Some embodiments of the invention can be achieved at different sizes or scales or size scales. Some components or parts or portions can be miniaturized. Dimensions can be macroscopic (as it is generally the case today), millimetric, microscopic, sub-microscopic if not at nano-scale.

In an embodiment, the glucose sensor tip size has the following dimensions (length of 13 mm to 14 mm; diameter at the base 0.20 to 0.30 mm; width 0.4 to 0.7 mm; degree of sensor insertion 45° to/or 90°). In an embodiment, the sensor tip is about 0.2 inches in length, about the thickness of a hair. In an embodiment, the sensor tip is connected to a water resistant, plastic on-body patch the size of a one-dollar coin. The sensor can remain inserted for 7 or 14 or 21 or 30 days and does not require finger stick calibrations (i.e. is “factory calibrated”). The sensor body (or “sensor patch”) connected to the tip has a compact form-factor (for example 35 mm×5 mm). In an embodiment, the reader device can only read data if held within 1.5 inches or the sensor patch. In other embodiments, data can be retrieved within tens of meters.

Microelectromechanical systems (MEMS) designate microscopic devices, possibly with moving parts. MEMS are also referred to as micro machines or micro systems technology (MST) in Europe. MEMS are made up of components between 1 and 100 micrometers in size (i.e. 0.001 to 0.1 mm), and MEMS devices generally range in size from 20 micrometers to a millimeter (i.e. 0.02 to 1.0 mm).

Nanotechnology (“nanotech”) designates the manipulation of matter on an atomic, molecular, and supramolecular scale (generally with at least one dimension sized from 1 to 100 nanometers). Nanoelectromechanical systems (NEMS) for example can use carbon-based materials as prime materials. Glucose nanosensors can be incorporated in implantable devices, advantageously enabling real-time tracking of blood glucose levels. In some embodiments, glucose-responsive nanoparticles can mimic the body's physiological needs for insulin. Nanotechnology enables oral insulin formulations, microspheres encapsulating islets, nanopumps, etc. Drug delivery focuses on maximizing bioavailability both at specific places in the body and over a period of time. Drug delivery can be achieved by molecular targeting by nanoengineered devices (e.g. efficient encapsulation of the drugs, delivery of drug to the targeted region of the body, effective release of the drug). Drug delivery systems for example can use lipid- or polymer-based nanoparticles, nanoparticles formed by the self-assembly of different microRNAs, phospholipids nano-particles, nanoelectromechanical systems (e.g. iron nanoparticles, gold shells). In an embodiment, nanotechnolgy is used to repair damages to the skin due to finger pricks (tissue engineering to help reproduce or repair or reshape damaged tissue using suitable nanomaterial-based scaffolds and growth factors). Nanoparticles such as graphene, carbon nanotubes, molybdenum disulfide and tungsten disulfide for example can be used.

In an embodiment, sensors are provided in the form of an injectable (or ingestible) nanoscale sensory network. Such a nanoscale sensory network can be “bioresorbable” (biodegradable in the bloodstream, dissolving after a few days, e.g. comprising biologically inert materials like silicon, or materials that won't cause an immune response or an overdose). Such a “nano-network” can degrade (spontaneously over time and/or can be controlled from the outside of the body, e.g. by an electromagnetic field and/or ultrasound) to release insulin when glucose levels are in excess to a predefined threshold (or a ranges of thresholds). In an embodiment, the nano-network can be formed of dextran nanoparticles loaded with insulin and glucose-specific enzymes. High glucose levels can activate these enzymes to convert glucose into gluconic acid, breaking down the dextran and releasing the insulin. In an embodiment, nanoparticles can be coated with negatively or positively charged film (to form the solid network). In some embodiments, a mixture of controllable nano sensors (e.g. measuring presence or concentration of one or more biomarkers) and nano actuators (e.g. controlling the release of one or more drugs, hormones, antigen, etc) can be injected. In some developments, the nano-network comprises locatable parts (to determine where the one or more releases of drugs shall occur within the body). An “image” or “map” of the patient body and the presence of sensors/actuators can be determined by processing means positioned outside the body. In an embodiment, the image or map is determined by consensus emerging from peer-to-peer exchanges and the decision to deliver drugs is performed without human intervention or open-loop. In some embodiments, an insulin pump can use micro-fluidics (synthesis of insulin, gene synthesis, etc).

In some embodiments, the glucose sensor can be about the thickness of a hair worn under the skin and connected to a water resistant, plastic on-body patch the size of a one-dollar coin. The sensor can remain inserted for 14 days and does not require finger stick calibrations (“factory calibrated”).

In some embodiments, the sensor size is between 10 and 15 mm in length (diameter at the base/tip from 0, 25 mm to 0, 5 mm). The degree of sensor insertion can range from 45 degrees to 90 degrees.

The flow of drug within the body can be facilitated in different ways. In an embodiment, nano or micro turbines can be used. Nanogenerators can use piezoelectric, triboelectric and/or pyroelectric nanogenerators.

### Analyte and/or Biomarker

In some embodiments, at least one sensor can determine the concentration of an analyte and/or of a biomarker.

Embodiments of the invention are applicable are applicable to humans and more generally to mammals (host).

Although the described examples are directed to a glucose sensor, the analyte sensor can be a sensor capable of determining the level of any suitable analyte in the body, for example, oxygen, lactase, insulin, hormones, cholesterol, medicaments, viruses, or the like.

Blood analyte or sample can be taken from capillary or interstitial or venous or arterial blood.

A diversity of blood analyte can be measured. The one or more analyte being measured can comprise one or more of a substance in a biological fluid, a chemical constituent in a biological fluid, a substance or chemical constituent in a biological fluid that can be analyzed, a substance in blood, a substance in interstitial fluid, a substance in lymph fluid, a substance in urine, an artificial substance, a metabolite, a reaction product.

An analyte can comprise acarboxyprothrombin, acylcarnitine, adenine phosphoribosyl transferase, adenosine deaminase, albumin, alpha-fetoprotein, amino acid profiles, arginine, histidineurocanic acid, homocysteine, phenylalaninetyrosine, tryptophan, andrenostenedione, antipyrine, arabinitol enantiomers, arginase, benzoylecgonine (cocaine), biotinidase, biopterin, c-reactive protein, carnitine, carnosinase, CD4, ceruloplasmin, chenodeoxycholic acid, chloroquine, cholesterol, cholinesterase, conjugated 1-.beta, hydroxy-cholic acid, cortisol, creatine kinase, creatine kinase MM isoenzyme, cyclosporin A, d-penicillamine, de-ethylchloroquine, dehydroepiandrosterone sulfate, DNA, acetylator polymorphism, alcohol dehydrogenase, alpha 1-antitrypsin, cystic fibrosis, DuchenneBecker muscular dystrophy, analyte-6-phosphate dehydrogenase, hemoglobin A, hemoglobin S, hemoglobin C, hemoglobin D, hemoglobin E, hemoglobin F, D-Punjab, beta-thalassemia, hepatitis B virus, HCMV, HIV-1, HTLV-1, Leber hereditary optic neuropathy, MCAD, RNA, PKU, Plasmodium vivax, 21-deoxycortisol, desbutylhalofantrine, dihydropteridine reductase, diptheriatetanus antitoxin, erythrocyte arginase, erythrocyte protoporphyrin, esterase D, fatty acidsacylglycines, free .beta.-human chorionic gonadotropin, free erythrocyte porphyrin, free thyroxine (FT4), free tri-iodothyronine (FT3), fumarylacetoacetase, galactosegal-1-phosphate, galactose-1-phosphate uridyltransferase, gentamicin, analyte-6-phosphate dehydrogenase, glutathione, glutathione perioxidase, glycocholic acid, glycosylated hemoglobin, halofantrine, hemoglobin variants, hexosaminidase A, human erythrocyte carbonic anhydrase I, 17-alpha-hydroxyprogesterone, hypoxanthine phosphoribosyl transferase, immunoreactive trypsin, lactate, lead, lipoproteins ((a), BA-1, .beta.), lysozyme, mefloquine, netilmicin, phenobarbitone, phenytoin, phytanicpristanic acid, progesterone, prolactin, prolidase, purine nucleoside phosphorylase, quinine, reverse tri-iodothyronine (rT3), selenium, serum pancreatic lipase, sissomicin, somatomedin C, etc.

An analyte also can comprise one or more of a metabolic product, an hormone, an antigen, an antibody and/or one or more trace elements (e.g. adenovirus, anti-nuclear antibody, anti-zeta antibody, arbovirus, Aujeszky's disease virus, dengue virus, Dracunculus medinensis, Echinococcus granulosus, Entamoeba histolytica, enterovirus, Giardia duodenalisa, Helicobacter pylori, hepatitis B virus, herpes virus, HIV-1, IgE (atopic disease), influenza virus, Leishmania donovani, leptospira, measlesmumpsrubella, Mycobacterium leprae, Mycoplasma pneumoniae, Myoglobin, Onchocerca volvulus, parainfluenza virus, Plasmodium falciparum, poliovirus, Pseudomonas aeruginosa, respiratory syncytial virus, rickettsia (scrub typhus), Schistosoma mansoni, Toxoplasma gondii, Trepenoma pallidium, Trypanosoma cruzirangeli, vesicular stomatis virus, Wuchereria bancrofti, yellow fever virus, specific antigen, hepatitis B virus, HIV-1 succinylacetone, sulfadoxine, theophylline, thyrotropin (TSH), thyroxine (T4), thyroxine-binding globulin, transferrin, UDP-galactose-4-epimerase, urea, uroporphyrinogen I synthase, vitamin A, white blood cell, zinc protoporphyrin, salt, sugar, protein, fat, vitamin, etc).

An analyte also can comprise a contrast agent for imaging, a radioisotope, a chemical agent, fluorocarbon-based synthetic blood, etc.

An analyte also can comprise one or more drugs and/or pharmaceutical compositions and/or stimulants and/or depressants and/or hallucinogens and/or neurochemical(ethanol, cannabis, marijuana, tetrahydrocannabinol, hashish, an inhalant, nitrous oxide, amyl nitrite, butyl nitrite chlorohydrocarbons, hydrocarbons, cocaine, crack, cocaine, meperidine, amphetamine, methamphetamine, phencyclidine, ecstasy, amphetamine, methamphetamine, Ritalin, Cylert, Preludin, Didrex, PreState, Voranil, Sandrex, Plegine, nicotine, barbituate, methaqualone, tranquilizer, Valium, Librium, Miltown, Serax, Equanil, Tranxene, phencyclidine, lysergic acid, mescaline, peyote, psilocybin, narcotic, heroin, codeine, morphine, opium, meperidine, Percocet, Percodan, Tussionex, Fentanyl, Darvon, Talwin, Lomotil, ascorbic acid, uric acid dopamine, noradrenaline, 3-methoxytyramine (3MT), 3,4-Dihydroxyphenylacetic acid (DOPAC), Homovanillic acid (HVA), 5-Hydroxytryptamine (5HT), 5-Hydroxyindoleacetic acid (FHIAA) etc)

The term “analyte” designates without limitation a substance or chemical constituent in a biological fluid (for example, blood, interstitial fluid, cerebral spinal fluid, lymph fluid or urine) that can be analyzed. Analyte can include naturally occurring substances, artificial substances, metabolites, and/or reaction products. Contemplated analytes include but are not limited to acarboxyprothrombin; acylcarnitine; adenine phosphoribosyl transferase; adenosine deaminase; albumin; alpha-fetoprotein; amino acid profiles (arginine (Krebs cycle), histidineurocanic acid, homocysteine, phenylalaninetyrosine, tryptophan); andrenostenedione; antipyrine; arabinitol enantiomers; arginase; benzoylecgonine (cocaine); biotinidase; biopterin; c-reactive protein; carnitine; carnosinase; CD4; ceruloplasmin; chenodeoxycholic acid; chloroquine; cholesterol; cholinesterase; conjugated 1-β hydroxy-cholic acid; cortisol; creatine kinase; creatine kinase MM isoenzyme; cyclosporin A; d-penicillamine; de-ethylchloroquine; dehydroepiandrosterone sulfate; DNA (acetylator polymorphism, alcohol dehydrogenase, alpha 1-antitrypsin, cystic fibrosis, DuchenneBecker muscular dystrophy, glucose-6-phosphate dehydrogenase, hemoglobin A, hemoglobin S, hemoglobin C, hemoglobin D, hemoglobin E, hemoglobin F, D-Punjab, beta-thalassemia, hepatitis B virus, HCMV, HIV-1, HTLV-1, Leber hereditary optic neuropathy, MCAD, RNA, PKU, Plasmodium vivax, sexual differentiation, 21-deoxycortisol); desbutylhalofantrine; dihydropteridine reductase; diptheriatetanus antitoxin; erythrocyte arginase; erythrocyte protoporphyrin; esterase D; fatty acidsacylglycines; free β-human chorionic gonadotropin; free erythrocyte porphyrin; free thyroxine (FT4); free tri-iodothyronine (FT3); fumarylacetoacetase; galactosegal-1-phosphate; galactose-1-phosphate uridyltransferase; gentamicin; glucose-6-phosphate dehydrogenase; glutathione; glutathione perioxidase; glycocholic acid; glycosylated hemoglobin; halofantrine; hemoglobin variants; hexosaminidase A; human erythrocyte carbonic anhydrase I; 17-alpha-hydroxyprogesterone; hypoxanthine phosphoribosyl transferase; immunoreactive trypsin; lactate; lead; lipoproteins ((a), BA-1, β); lysozyme; mefloquine; netilmicin; phenobarbitone; phenytoin; phytanicpristanic acid; progesterone; prolactin; prolidase; purine nucleoside phosphorylase; quinine; reverse tri-iodothyronine (rT3); selenium; serum pancreatic lipase; sissomicin; somatomedin C; specific antibodies (adenovirus, anti-nuclear antibody, anti-zeta antibody, arbovirus, Aujeszky's disease virus, dengue virus, Dracunculus medinensis, Echinococcus granulosus, Entamoeba histolytica, enterovirus, Giardia duodenalisa, Helicobacter pylori, hepatitis B virus, herpes virus, HIV-1, IgE (atopic disease), influenza virus, Leishmania donovani, leptospira, measlesmumpsrubella, Mycobacterium leprae, Mycoplasma pneumoniae, Myoglobin, Onchocerca volvulus, parainfluenza virus, Plasmodium falciparum, poliovirus, Pseudomonas aeruginosa, respiratory syncytial virus, rickettsia (scrub typhus), Schistosoma mansoni, Toxoplasma gondii, Trepenoma pallidium, Trypanosoma cruzirangeli, vesicular stomatis virus, Wuchereria bancrofti, yellow fever virus); specific antigens (hepatitis B virus, HIV-1); succinylacetone; sulfadoxine; theophylline; thyrotropin (TSH); thyroxine (T4); thyroxine-binding globulin; trace elements; transferrin; UDP-galactose-4-epimerase; urea; uroporphyrinogen I synthase; vitamin A; white blood cells; and zinc protoporphyrin. Salts, sugar, protein, fat, vitamins and hormones naturally occurring in blood or interstitial fluids can also constitute analytes in certain embodiments. The analyte can be naturally present in the biological fluid, for example, a metabolic product, a hormone, an antigen, an antibody, and the like. Alternatively, the analyte can be introduced into the body, for example, a contrast agent for imaging, a radioisotope, a chemical agent, a fluorocarbon-based synthetic blood, or a drug or pharmaceutical composition, including but not limited to insulin; ethanol; cannabis (marijuana, tetrahydrocannabinol, hashish); inhalants (nitrous oxide, amyl nitrite, butyl nitrite, chlorohydrocarbons, hydrocarbons); cocaine (crack cocaine); stimulants (amphetamines, methamphetamines, Ritalin, Cylert, Preludin, Didrex, PreState, Voranil, Sandrex, Plegine); depressants (barbituates, methaqualone, tranquilizers such as Valium, Librium, Miltown, Serax, Equanil, Tranxene); hallucinogens (phencyclidine, lysergic acid, mescaline, peyote, psilocybin); narcotics (heroin, codeine, morphine, opium, meperidine, Percocet, Percodan, Tussionex, Fentanyl, Darvon, Talwin, Lomotil); designer drugs (analogs of fentanyl, meperidine, amphetamines, methamphetamines, and phencyclidine, for example, Ecstasy); anabolic steroids; and nicotine. The metabolic products of drugs and pharmaceutical compositions are also contemplated analytes. Analytes such as neurochemicals and other chemicals generated within the body can also be analyzed, such as, for example, ascorbic acid, uric acid, dopamine, noradrenaline, 3-methoxytyramine (3MT), 3,4-dihydroxyphenylacetic acid (DOPAC), homovanillic acid (HVA), 5-hydroxytryptamine (5HT), and 5-hydroxyindoleacetic acid (FHIAA).

The frequency of measurements can be variable, for example contextual (e.g. repeated if a risk of hypoglycemia is higher than a predefined threshold), can depend on the current uncertainty of associated algorithms.

In some embodiments of the invention, the medical system comprises a monitoring device responsible for the detection of a particular analyte. The sensing region generally comprises a non-conductive body, a working electrode (anode), a reference electrode (optional), and/or a counter electrode (cathode) passing through and secured within the body forming electrochemically reactive surfaces on the body and an electronic connective means at another location on the body, and a multi-domain membrane affixed to the body and covering the electrochemically reactive surface.

### Minimally-Invasive or Non-Invasive Sensors

In some embodiments, at least one sensor can be minimally-invasive or non-invasive.

In some embodiments, the medical system according to the invention comprises a non-invasive monitoring device, e.g. a breathalyzer. The breathalyzer comprises a chamber containing a test slide (which can be one-use or allow multiple uses). The slides are for example coated with a nanometer-thick film, comprising two or more polymers that react with acetone. The slide is then read out and the level of acetone is determined. In other embodiments, the presence of other biomarkers is determined. Data can be taken into account to improve the evaluation of blood glucose level.

In some embodiments, nano sensors can comprise biological or artificial receptors for glucose which can transduce glucose concentrations into changes in fluorescence. For example, nano sensors can comprise lectins (e.g. plant lectin concanavalin-A) and/or enzymes (e.g. hexokinase) and/or bacterial binding proteins (e.g. Glucose/Galactose-Binding Protein (GBP), boronic acid derivatives). The layer-by-layer (LBL) technique can be used. Quantum dots can be used. Carbon nanotubes can be used (e.g. for continuously measuring the transfer of electrons produced when insulin molecules oxidize in the presence of glucose). Nanoparticles can endorse an anti oxidative role in diabetes (e.g. Cerium oxide, Yttrium oxide, alumina, Silver nitrate, AuNPs).

### Implantable Sensors

In some embodiments, at least one sensor and/or actuator is implementable.

In some embodiments of the invention, the medical system comprises a transcutaneous analyte sensor system which includes an applicator for inserting the transdermal analyte sensor under a host's skin. The sensor system includes a sensor for sensing the analyte, wherein the sensor is associated with a mounting unit adapted for mounting on the skin of the host. The mounting unit houses the electronics unit associated with the sensor and is adapted for fastening to the host's skin. In certain embodiments, the system further includes a receiver for receiving and/or processing sensor data.

In some embodiments, one or more sensors and/or one or more drug delivery actuators can be embedded in one or more artificial implantable teethes. Teethes can represent one or more available “volumes” for instrumentation which can be advantageously leveraged (for adults, in particular molars and pre-molars). In an embodiment, an artificial implantable/implanted tooth comprises mechanisms for fluid extraction and/or analysis of the “gingival crevicular fluid” which has glucose levels very close to plasma (in particular, the “pulp” inside teeth is extensively vascularized, with high rates of blood flow and high blood pressure). Glucose measurements (and/or uric acid levels, biomarkers, etc) can be performed ithin the one or more artificial teethes and/or in an external device introduced into the mouth to perform fluid sample extraction. For example, an on-chip disposable enzyme-based nano-biosensor can be used. Real-time salivary glucose tracking or mouth activity can advantageously complement other BG monitoring (e.g. carbs intake determined passively can modulate algorithms for closed-loop artificial pancreas, such as hypoglycemia prediction algorithms). In some embodiments, as many artificial teethes can be used, a sufficient volume of basal insulin and/or fast insulin can be rendered available (e.g. nominal operation or for fallback). Other drugs also can be used (e.g. anti-toxin, antivenom or antivenin or antivenene, anti-poison, glucagon, epinephrine to survive anaphylaxis, antibiotics or antiviral agents, etc). Microfluidics and other miniaturized delivery mechanisms can be embedded, i.e. within one or more teethes. Other sensors can be embedded: for example, an accelerometer (e.g. dynamo and/or induction powered) can determine the masticary quantity/intensity to assess carbs intake (e.g. by measuring jaw movement, and categorizing different activities of the mouth). A microphone also can be used. Bone conduction can allow sound restitution in some cases. A memory unit can store critical data (e.g. medical condition, access credentials, etc). In some embodiments, one or more sensors and/or one or more drug delivery actuators can be releasable (e.g. removable, disposable, etc). For example, an artificial dental root/neck can serve as a support for a disposable sensor (e.g. in the shape of a dental crown). Connection can be mechanical (complimentary pieces) and/or chemical (e.g. glue) and/or electrochemical and/or magnetic, etc. Different configurations and thus therapeutic schemes (implemented in associated software) can be allowed if a plurality of releasable modular implants is used. One or more of the preceding elements or devices or apparatus can be controlled externally and/or remotely, unidirectionally or bidirectionnaly (command/action). Previously described security mechanisms can be used.

In some embodiments, the medical system (according to any one of the presently described embodiments) can comprise one or more encapsulating devices.

Encapsulating devices can comprise “immuno-isolatory” devices, which when implanted into a mammalian host, can minimize the deleterious effects of the host's immune system on the cells within the core of the device. The surrounding or peripheral region of the device can confer protection to encapsulated cells from the immune system of the host in whom the device or assembly is implanted, prevent harmful substances of the host's body from entering the device, and provide a physical barrier sufficient to prevent detrimental immunological contact between the isolated cells and the immune system of the host. The thickness of the physical barrier can vary, but it will always be sufficiently thick to prevent direct contact between the cells and/or substances on either side of the barrier. The thickness of this region generally can range between 5 and 200 microns; a thickness of 10 to 100 microns is preferred, and thickness of 20 to 75 microns is particularly preferred. Types of immunological attack which can be prevented or minimized by the use of the instant vehicle include, but are not limited to, attack by macrophages, neutrophils, cellular immune responses (e.g., natural killer cells and antibody-dependent T cell-mediated cytolysis (ADCC)), and humoral response (e.g., antibody-dependent, complement-mediated cytolysis).

In some embodiments, encapsulating devices can comprise a semi-permeable membrane which can allow the encapsulated biologically active substance of interest to pass (e.g., insulin, glucagon, pancreatic polypeptide and the like), making the active substance available to the target cells outside the device and in the patient's body. In an embodiment, the permeability can be configurable or controllable.

Encapsulating devices can comprise of a biocompatible material including, but are not limited to anisotropic materials, polysulfone (PSF), nano-fiber mats, polyimide, tetrafluoroethylene/polytetrafluoroethylene (PTFE; also known as Teflon®), ePTFE (expanded polytetrafluoroethylene), polyacrylonitrile, polyethersulfone, acrylic resin, cellulose acetate, cellulose nitrate, polyamide, as well as hydroxylpropyl methyl cellulose (HPMC) membranes.

Encapsulating devices can contain a plurality of chambers or compartments (better capable to disperse the cells throughout the chamber/compartment or chambers/compartments, more opportunity for each cell to receive nutrients and oxygen, etc)

In an embodiment, devices or assemblies are expandable. In one embodiment, encapsulation devices can comprise a refillable reservoir, lumen, container or compartment, which can be periodically filled or flushed with appropriate therapeutic or biologically active agents and/or cells. In an embodiment, encapsulation devices can comprise luminal or chamber matrix, foam or scaffold or insert between the walls of the cell encapsulating device forming the cell chamber.

Imaging methods associated with encapsulating devices can include confocal microscopy, 2-photon microscopy, high and low frequency ultrasound, optical coherence tomography (OCT), photoacoustic tomography (PAT), computed tomography (CT), magnetic resonance imaging (MRI), single photon emission computed tomography (SPECT) and positron emission tomography (PET).

### Other Embodiments

In some embodiments, the medical system according to the invention can comprise one or more contact lens and/or a spectrometer and/or a drone and/or a wearable computer.

Said macro-objects can embed said sensors and/or actuators. Multiple devices can enable the cooperative calibration of different devices.

In some embodiments, one or two contact lens can be worn by the user. A contact lens can comprise biosensors and/or a pulse oximetry sensor and/or display means (indicating an hypoglycemia or hyperglycemia or risk thereof), as well as other micron-scale devices (photoreceptors, LEDs, etc).

A (smart) contact lens can comprise miniaturized electronics (micron-scale devices) e.g. one or more integrated biosensors configured to test for presence of one or more biomarkers bound to one or more receptors disposed in one or more cavities formed at predetermined locations within a body of a contact lens for determining state information associated with an individual from which the biomarkers were generated.

The smart contact lens can be connected to other devices (NFC communication, micro-antennas). An annular antenna (or a network of antennas) can be disposed at a margin of the contact lens, wherein the antenna is configured to both receive a power signal and transmit a data signal. A biosensor can comprise an electromechanical sensor comprising a working electrode, a counter electrode and a reference electrode. The contact lens can comprise a communications module configured to process the power signal from the antenna to provide operational power to the biosensor and process the biosensor signal to provide the data signal to the antenna (e.g. using backscatter modulation). The tear fluid generated by the individual can be continuously or intermittently monitored, using micro fluidics and MEMS (extraction components configured to extract the tear fluid comprising the one or more biomarkers bound to one or more receptors disposed in the one or more cavities without disrupting bonds between the one or more biomarkers and the one or more receptors or biosensors, rinsing compartments, etc). The dye can react or bind with a selected bioanalyte present in tears such that reacting or binding of the bioanalyte is associated with a detectable change in optical properties of the dye (by the person wearing the contact lens and/or my a camera monitoring color changes). Regarding the structure, the contact lens can comprise a plurality of cavities (e.g. forming an intricate network of canals and/or cells, for example optimizing capillary surfaces and/or gravity) receiving a plurality of receptors or detector molecules or biosensors (e.g. with openings on the inner or the outer surface of the contact lens). Where the substrate has a thickness of about 5000 μm, a cavity can have a width or depth of about 500 μm or less. Integrated biosensors can be located at positions such as to not directly obstruct the vision of the person wearing the contact lens.

Detector molecules can use an antibody covalently linked to an enzyme, a detector antibody configured to bind to the one or biomarkers bound to the one or more receptors, and another detector molecule comprising a substrate configured to bind to the enzyme to produce a signal. The state information can include at least one of a glucose level, alcohol level, histamine level, urea level, lactate level, cholesterol level, sodium ion level, potassium ion level, calcium ion level or magnesium ion level of the individual. A receptor can include a biological or chemical component having a binding site for a known ligand. A receptor can include but is not limited a biomolecule (including proteins, peptides, polysaccharides, lipids, hormones and nucleic acids as well as small molecules such as primary metabolites, secondary metabolites, and natural products), an antibody, an antibody linked with an enzyme, an antigen, or a synthetic molecule. The term ligand refers to a molecule having a known binding affinity for a known receptor. A ligand designates a molecule which binding properties are to be analyzed. A ligand can include a chemical, a biomolecule, a complex organism (e.g. human pathogen) a pharmaceutical drug, a toxin, an antigen or an antibody. Ligands can also include airborne molecules and chemicals including but not limited to pollutants, allergens, viruses, or bacteria. Receptors can be employed that bind to known ligands that serve as biomarkers. A biomarker refers to a biological molecule or substance which can be used to indicate a biological state. Biomarkers can be objectively measured and evaluated as indicators of normal biological processes, pathogenic processes, or pharmacologic responses to a therapeutic intervention. A biosensor can include a physiologically compatible oxygen substrate (e.g. transparent and flexible materials such as PDMS or silicone acrylate, silicone derivative, polyacrylate and fluorofoether) comprising porous nanostructures (e.g. zeolite materials, for example aluminosilicate nanocomposite zeolites adhered on said substrate, said nanostructures comprising a physiologically compatible fluorescent assay containing at least one physiologically compatible fluorescent dye or detector molecules(e.g. FITC Dextran-TRITC-Con A, wherein the bioanalyte being detected for is glucose present in tears) encapsulated therein. The nanostructures can comprise silica nanoparticles, nanotubes, nanofilms, and bio-polymer nanostructures including alginate, chitosan nanoparticles (NP), nanofibers, 2-D and 3-D foams with highly nanoprorous structures. The fluorescent dye can be FITC Dextran-TRITC-Con A or FITC Dextran-TRITC-Con A or tetramethyl rhodamine isothiocyanate (TRITC) and 9,10-diphenyl anthracene or a pair of Fluorophor 1—Protein (Con A)—Fluorophor2, wherein the pair of Fluorophor 1 and Fluorophor 2 include rhodamine and fluorescein isothiocyanate (FITC), tetramethyl rhodamine isothiocyanate (TRITC), and fluorescein isothiocyanate (FITC), or tetramethylrhodamine (TAMRA) and FITC (FITC-dextran). The physiologically compatible fluorescent assays can be malachite green (MG) and crystal violet (CV). The biosensor can include transparent micro/nanospheres having a diameter in a range from about 20 nm to about 200 nm, said transparent micro/nanospheres containing any one or combination of drugs, artificial tears, and cooling agents to reduce symptoms of dry eyes. The micro/nanospheres can be made from materials selected from the group consisting are PLGA, collagen, hydrogels, and alginate. The porous structures can be selected from the group consisting of mesoporous silica nanomaterial, hollow tubes having nano/micro-scale dimensions, fibers having nano/micro-scale dimensions, and porous polymer spheres having nano/microscale dimensions.

In addition to biosensors, a contact lens can comprise a pulse oximetry sensor located on or within the substrate configured to detect information associated with at least one of a blood oxygen content or a pulse rate of a wearer of the contact lens, the pulse oximetry sensor comprising: one or more light emitting diodes configured to illuminate a blood vessel of at least one of a region of an eye or an eyelid; and a detector configured to receive light transmitted through the blood vessel and generate the information, wherein the information includes a signal indicating an amount of light transmitted through the blood vessel; wherein the one or more light emitting diodes and the detector are positioned away from a center of the contact lens; the contact lens configured to maintain an orientation when worn on an eye such that the one or more light emitting diodes and the detector are not covered by an eyelid when the eye is open.

In an embodiment, the medical system comprises at least one contact lens configure to measure and/or evaluate glucose values. The glucose concentration of the aqueous humor can at most one one-hundredth as fast as that of the blood. There is generally a delay of about 45 minutes to one hour between a measurement of glucose in blood and a valid reading of a changed glucose value in the anterior chamber.

In an embodiment, the contact lens or smart watch comprises optical and acoustic transducers which are coupled to tissue in a manner which permits blood analytes measurements to be made. In some embodiments, a quantum cascade laser is arranged with crystalline acoustic detectors in a photo acoustic effect measurement scheme. Laser pulses stimulate special vibrational states of glucose molecules to produce an acoustic return signal to be received at a piezoelectric detector. A wristwatch case may include a back member which supports arrangements and coupling between the back of the watch, elements contained therein, and tissue in contact with the device.

In an embodiment, the medical system comprises an implementation of steps comprising illuminating the eye from two or more, different-wavelength light sources whose respective wavelengths interact with internal eye properties in optically differentiated manners, adjusting the operating levels of the sources to a predetermined relative setting, producing seriatim-light-source eye reflections including multiple internal reflections within the outer structure of the eye, and at least one resulting outbound reflection, monitoring the outbound-reflection to detect therein the relative reflection levels associated with the sources, and associating said detected, relative reflection levels with at least one eye property. In an embodiment, an eye property is associated with the apparent thickness change of the volume of the corneal tissue and/or variations in refractive index of the aqueous humor and/or measuring optically thickness variations of the cornea to determine glucose, said parameters being associated with a blood-glucose concentration level.

In an embodiment, non-invasive measurements of the glucose concentration use a combination of differential scattering spectroscopy and confocal scanning laser Doppler microscopy.

Data signals can be transmitted and analyzed by computing devices of the user (i.e. smartphone or smart watch) but a contact lens can restitute graphical information to the user (graphical dots, arrays of dots, if not displays). Graphical indicators can be displayed to the user wearing one or two contact lens in an augmented reality manner (the placement of the indicator can be placed at optimized location by superposition in the field of view of the user). A “spatial grammar” (universal or specific to each person) can optimize the use of the patient's cognitive attention (for example a specific user may want the sky to change colors through the contact lens, indicating BG levels e.g. nuances from dark to light blue). In some embodiments, a plurality of contact lens can be superposed (additive functions).

In an embodiment a micro UAV or drone can inject insulin or at least provide the user with the insulin pen. The drone can be mind-controlled and/or be fully autonomous. In an embodiment, a personal robot (personal computer with displacement capabilities) can bring injection and/or measurement devices.

For example, a micro drone for drug delivery can use radar and/or a laser anemometer to deliver insulin (or anti-toxin). An electronic nose (olfactive sensors) can be used to evaluate blood glucose. A nano or micro device in bloodstream can use a SONAR device. A glass break or flame detector can be used to secure an insulin pump. Lasers can be used to puncture skin.

Sophisticated sensors can be used on the battlefield and/or critical (e.g. radioactive) environments.

In an embodiment, massage (moving and/or rotating) pieces can be provided. Massage of the superficial layers of the skin advantageously can help to improve the diffusion of insulin from the insulin depot having being injected. Massage or more generally movements of fluid under the skin can be facilitated in several ways. For example, magnetic (bio compatible or evacuable) particles can be used in combination with magnetic guides. Mechanical massage can be used (e.g. with rollers). Eletrical means can be used. Massage also can be ultrasonic. Electro-mechanical devices and associated sensors can be used to deliver massages to the skin to facilitate insulin diffusion from insulin depot.

In an embodiment, the “scan” operation (e.g. NFC reading step) can be performed by a drone or micro-drone flying in the room and seeking to retrieve data out of the FGM or modified FGM. The “scan” also can be executed incidentally, i.e. when the patient passes by NFC reading devices affixed in the living space.

The medical system can comprise one or more spectrometers. Food scanners can for example communicate how many and what kind of ingredients, how many allergens, toxins, how many carbohydrates a given food actually contains.

Near-IR spectroscopy can be used (food analysis for evaluating carbs, tissue analysis, etc). Spectrometric models can translate measures into calorie counts, percentage of carbohydrates, fat, and protein contained in the food, for example. Volume analysis can be estimated by machine vision (and/or by manual measurement, for example with a scale). Bolus values can be proposed based on volumes and carbs by volume information (and patient profile or therapy).

Near-infrared spectroscopy (NIRS) is a spectroscopic method that uses the near-infrared region of the electromagnetic spectrum (from about 700 nm to 2500 nm).

NIR can typically penetrate much farther into a sample than mid infrared radiation. Silicon-based CCDs can be used. InGaAs and PbS devices can be used. Optical Coherence Tomography (OCT) as a NIR medical imaging technique can allow 3D imaging with high resolution on par with low-power microscopy. By using optical coherence to measure photon path length, images of live tissue or tissue morphology can be determined (for example insulin depot and diffusion can be analyzed).

A compact spectrometer system for obtaining the spectrum of a sample, can comprise an optical detector for detecting light emanating from said sample; an optical filter located between said sample and said detector; and a first Fourier transform focusing element wherein said compact spectrometer system does not contain any dispersive optical elements. The optical filter can be a non-tunable filter. The first Fourier transform focusing element can be disposed between said optical filter and said optical detector such that light passing through said optical filter is dispersed by said at least one focusing element onto the light-sensitive surface of said detector. The center wavelength of the optical filter varies with the incidence angle of light impinging thereupon. The optical filter can comprise a plurality of sub-filters with different center wavelengths. The optical filter can comprise a plurality of substantially parallel strips, each of which comprises a sub-filter. The optical filter can comprise a plurality of substantially rectangular areas, each of which comprises a sub-filter. The optical filter can be chosen from the group consisting of (a) Fabry-Perot filter, (b) thin-film filter, and (c) interference filter. The first Fourier transform focusing element can be a plano-convex lens disposed such that its flat face faces said optical detector and its curved face faces said optical filter. The compact spectrometer can further comprise a second Fourier transform focusing element. The Fourier transform focusing elements can be plano-convex cylindrical lenses disposed such that the flat face of each lens faces said optical detector; the curved face of each lens faces said optical filter; the focal lines of the two lenses are oriented along different axes in the x-y plane; and, the focal planes of said Fourier transforming focusing elements substantially coincide. The focal planes of said Fourier transforming focusing elements can be substantially coincident with light-sensitive surface of said optical detector. The focal lines of said Fourier transform focusing elements can be perpendicular. The compact spectrometer system can further comprise a micro-lens array. The micro-lens array can be located in the focal plane of said first Fourier transform focusing element. The detector can be located at a plane substantially perpendicular to the optical axis such that the micro-lenses form multiple images of said optical filter on said optical detector. The optical filter can comprise a plurality of sub-filters with different center wavelengths. The compact spectrometer system can further comprise a second Fourier transforming focusing element, wherein said micro-lens array comprises an array of cylindrical lenses and is located at the focal plane of first of two said focusing elements and said optical detector is located at the focal plane of second of two said focusing elements. The compact spectrometer system can further comprise a diffuser disposed between said sample and said optical filter. The first Fourier transform focusing element can be a lens chosen from the group consisting of (a) plano-convex lenses, (b) biconvex lenses, and (c) aspheric lenses, and further wherein said optical filter is located between said first Fourier transform focusing element and said sample. The optical filter comprises a plurality of sub-filters with different center wavelengths

The plurality of sub-filters is disposed radially about a center point. The optical filter can be in close proximity to said optical detector. The optical detector can be a two-dimensional image sensor. The compact spectrometer system can further comprise a light source adapted to illuminate said sample. The light source can be a laser. The light source can be a light-emitting diode. The compact spectrometer system can further comprise a focusing system adapted focus light from said light source at a predetermined location relative to said sample. The focusing system can be an autofocus system. The focusing system can control the position of a lens that focuses light produced by said light source onto said sample. The focusing system can control the optical properties of a lens that focuses light produced by said light source onto said sample. The focusing system can comprise a voice-coil motor. The focusing system can comprise a piezoelectric motor. The focusing system can comprise a micro-electrical-mechanical-system (MEMS) motor. The light emanating from said sample can comprise light scattered by said sample upon illumination.

The spectrum can be selected from the group consisting of (a) molecular vibrational spectra, (b) molecular rotational spectra, and (c) electronic spectra. The spectrum can be a Raman spectrum. The compact spectrometer system can further comprise a second optical filter. The light scattered from said sample upon illumination can comprise light reflected by said sample upon illumination. The light emanating from said sample can comprise light produced by fluorescence emanating from said sample. The compact spectrometer system can further comprise means for communicating with a communication network. The compact spectrometer system can be enclosed within a mobile communication device associated with said communication network. The compact spectrometer system is a cellular telephone or a smartphone. The compact spectrometer system can be incorporated into head-mounted display, or smartglasses, or a smartwatch or an oven, such as a microwave oven, or into a refrigerator. The sample can comprises food.

In some embodiments, drug delivery means and/or analyte sensing means can use smart textile (e.g. flexible electronics). Embodiments of the invention can comprise one or more “e-textile” devices.

“E-textile” or “smart garments” or “smart clothing” or “electronic textile” or “smart textiles” or “smart fabrics” or “textronics” or “fibretronics” designate fabrics that enable digital components (including small computers) and electronics to be embedded in them. Electronic textiles (e-textiles) are generally fabrics which have electronics and interconnections woven or otherwise integrated into them. E-textiles generally present physical flexibility. E-textiles can integrate sensors, microchips and/or other devices. E-textile embodiments designate hardware and/or software embodiments. Software designates information processing (such as fault tolerance in light of manufacturing defects and quality of service) within the e-textile and/or between the e-textile and external agents/devices.

An e-textile device can comprise “stretchable electronics”, which designate elastic electronics or elastic circuits (e.g. obtained by depositing stretchable electronic devices and circuits onto stretchable substrates or embed them in a stretchable material such as silicones or polyurethanes). Stretchable electronics can comprise elastic PDMS substrates, buckled SWCNTs macrofilm and elastomeric separators. An elastic microsystem can be divided into functional islands (comprising electronic components), which are interconnected by stretchable interconnects. The whole can be encapsulated into an elastic polymer. Stretchable interconnections for example can be obtained by embedding meander shaped wires in an elastic base material.

An e-textile device can comprise “flexible electronics” (electronic devices mounted on flexible plastic substrates, such as polyimide, PEEK or transparent conductive polyester film). Flexible circuit structures can comprise single-sided flex circuits, double access or back bared flex circuits, sculptured flex circuits, double-sided flex circuits, multilayer flex circuits, polymer thick film flex circuits, etc. Flexible circuit materials can comprise base material comprising polyester (PET), polyimide (PI), polyethylene naphthalate (PEN), polyetherimide (PEI), along with various fluropolymers (FEP) and copolymers, one or more bonding adhesives and foils (e.g. metal).

An e-textile device can comprise electronic ink, Gyricon and/or OLED. An e-textile device can comprise smart dyes, nanofibers, drug-releasing fibers, light emitting fabrics, etc. Conductive inks can be used. Electroluminescence can be used.

An e-textile device can present different arrangements, e.g. layers and/or arrays and/or graphs and/or meshes and/or foams and/or (macro, micro, nano) springs, foldings (e.g. Origami) etc.

Fabric sensors can be used for electrocardiogram (ECG), electromyography (EMG), electroencephalography (EEG) sensing. Fabrics incorporating thermocouples can be used for sensing temperature. Luminescent elements integrated in fabrics can be used for biophotonic sensing. Shape-sensitive fabrics can sense movement, and can be combined with EMG sensing to derive muscle fitness. Carbon electrodes can be used to detect specific environmental or biomedical features such as oxygen, salinity, moisture, or contaminants.

For example, a “smart shirt” can comprise a T-shirt wired with optical and conductive fibers to collect biomedical information, for example integrating sensors for monitoring the signs such as heart rate, respiration rate, electrocardiogram (ECG), pulse oximetry and temperature, among others. For example, “smart socks” can comprise built-in pressure sensors to detect poor blood circulation. A “smart bra” can change its properties in response to breast movement (e.g. a polymer fabric to expand and contract in response to movement). Some other devices can comprise ionic biosensors, for example capable of measuring sodium, potassium and chloride in sweat samples. Some probes can measure the conductivity of sweat. A pH sensor can use color changes (e.g. with a portable spectrometer device) to indicate the pH of sweat. An immunosensor can detect the presence of specific proteins in fluid samples. Reflective oximetry can be used to measure levels of oxygen saturation in the blood (e.g. around the thorax). Combination or patterns of hydrophilic and hydrophobic yarns can collect sweat for further analysis.

An e-textile device (e.g. shirt, pants, socket, belt, etc.) can comprise electrical conductive fibers (e.g. ferrous alloys, nickel, stainless steel, titanium, aluminum, copper, carbon, etc.). An e-textile device can comprise optical conductive fibers (e.g. perfloro polymers, molten glass in filaments, etc.). An e-textile device can comprise organic electronics materials (conducting or semiconducting). An e-textile device can comprise conducting lines or fibers or links designed as inks and/or plastics. An e-textile device can comprise wired and/or wireless connections (data from socks can be communicated to processing units located near the chest for example). In some cases, the electrical conductivity of the skin can be leveraged for data communication (or verification or appairing etc.).

An e-textile device can comprise wire electrochemical transistor devices and textile monofilaments which can be coated with continuous thin films of conducting matter (e.g. polythiophene poly(3,4-ethylenedioxythiophene). Three-dimensional polymer micro-electronics can be used.

An e-textile device can present photovoltaic capabilities. For example, an e-textile device can comprise a phase-separated, photovoltaic layer, comprising a conducting polymer and a fullerene derivative, which can be coated onto a thin metal wire. A second wire, coated with a silver film, serving as the counter electrode, can be wrapped around the first wire. Both wires can be encased in a transparent polymer cladding. Incident light is then focused by the cladding onto to the photovoltaic layer even when it is entirely shadowed by the counter electrode.

An e-textile device can incorporate components into the textile structure by different technologies (e.g. embroidering, sewing, non-woven textile, knitting, spinning, breading, coating/laminating, printing and chemical treatment).

An e-textile device can use micro-device encapsulation technology to encapsulate devices with a flexible hermetic seal for mechanical, thermal and electrical protection. To avoid damages during washing, at least some parts of the e-textile device can be removable/releasable (e.g. one or more textile patches). Damaged circuits can be self-healed or repaired by using particular data routing (peer-to-peer or mesh network).

Regarding energy, different sources or energy can be used and combined (e.g. battery, zn-air, kinetic energy, stretch energy, dynamo, solar cells, micro-springs, etc.).

An e-textile device can be used to display for example by embedding micro-LEDs. Flexible displays and “e-textile” can converge and allow to increase the surface available for display of information (textual and/or visual e.g. temperature of the body). Micro-turbines can cool down embedded processors and/or optimize body heat for comfort or pleasure). An e-textile device can comprise video display devices such as Organic light-emitting diode (OLED), AMOLED Organic light-emitting transistor (OLET), Surface-conduction electron-emitter display (SED), Field emission display (FED), Laser TV Quantum dot, Liquid crystal, MEMS display, IMoD, TMOS, DMS Quantum dot display (QD-LED), Ferro liquid crystal display (FLCD), Thick-film dielectric electroluminescent technology (TDEL), Telescopic pixel display (TPD), Laser-powered phosphor display (LPD), etc. An e-textile device can comprise non-video display devices such as Electromechanical (Flip-dot, Split flap Vane), Eggcrate, Nixie tube, Vacuum fluorescent display (VFD), Light-emitting electrochemical cell (LEC), Lightguide display, Dot-matrix display, Seven-segment display (SSD), Fourteen-segment display (FSD), Sixteen-segment display (SISD), etc.

In some advantageous embodiments of the invention, sensors (e.g. blood glucose sensor) can be inserted in the scalp (head skin), more precisely in the connective tissue (which is a subcutaneous layer containing the nerves and vessels of the scalp). Hairs can advantageously hide a patch with sensors. The scalp presents a large addressable surface for sensing and/or injecting. The blood supply of the scalp is performed via five pairs of arteries, three from the external carotid and two from the internal carotid. The blood supply is advantageous for blood analysis.

In some advantageous embodiments of the invention, sensors (e.g. blood glucose sensor) and/or actuators can be inserted in one or two earlobes. An earlobe does not contain cartilage and generally presents a large blood supply. Drug reservoir can be hidden into the ear (outer ear e.g. concha i.e. cavum and/or cymba, behind the ear, etc). Piercing-shapes devices can be used.

In some embodiments, the medical system (according to any one of the presently described embodiments) can comprise one or more food-identifying (and/or classifier) sensors or devices.

In an embodiment, the medical system can comprise a food-identifying sensor (e.g. image segmentations and comparisons, image matching, classifiers etc). Such a sensor or device or implemented logic can detect or measure a selected food, ingredient, or nutrient that has been designated as unhealthy by a health care professional organization or by a specific health care provider for a specific person; a selected substance that has been identified as an allergen for a specific person; peanuts, shellfish, or dairy products; a selected substance that has been identified as being addictive for a specific person; alcohol; a vitamin or mineral; vitamin A, vitamin B1, thiamin, vitamin B12, cyanocobalamin, vitamin B2, riboflavin, vitamin C, ascorbic acid, vitamin D, vitamin E, calcium, copper, iodine, iron, magnesium, manganese, niacin, pantothenic acid, phosphorus, potassium, riboflavin, thiamin, and zinc; a selected type of carbohydrate, class of carbohydrates, or all carbohydrates; a selected type of sugar, class of sugars, or all sugars; simple carbohydrates, complex carbohydrates; simple sugars, complex sugars, monosaccharides, glucose, fructose, oligosaccharides, polysaccharides, starch, glycogen, disaccharides, sucrose, lactose, starch, sugar, dextrose, disaccharide, fructose, galactose, glucose, lactose, maltose, monosaccharide, processed sugars, raw sugars, and sucrose; a selected type of fat, class of fats, or all fats; fatty acids, monounsaturated fat, polyunsaturated fat, saturated fat, trans fat, and unsaturated fat; a selected type of cholesterol, a class of cholesterols, or all cholesterols; Low Density Lipoprotein (LDL), High Density Lipoprotein (HDL), Very Low Density Lipoprotein (VLDL), and triglycerides; a selected type of protein, a class of proteins, or all proteins; dairy protein, egg protein, fish protein, fruit protein, grain protein, legume protein, lipoprotein, meat protein, nut protein, poultry protein, tofu protein, vegetable protein, complete protein, incomplete protein, or other amino acids; a selected type of fiber, a class of fiber, or all fiber; dietary fiber, insoluble fiber, soluble fiber, and cellulose; a specific sodium compound, a class of sodium compounds, and all sodium compounds; salt; a selected type of meat, a class of meats, and all meats; a selected type of vegetable, a class of vegetables, and all vegetables; a selected type of fruit, a class of fruits, and all fruits; a selected type of grain, a class of grains, and all grains; high-carbohydrate food, high-sugar food, high-fat food, fried food, high-cholesterol food, high-protein food, high-fiber food, and high-sodium food. In an example, a device for measuring or estimating (number of mastication's) a person's consumption of at least one specific food, ingredient, and/or nutrient that can analyze food composition can also identify one or more potential food allergens, toxins, or other substances for example: ground nuts, tree nuts, dairy products, shell fish, eggs, gluten, pesticides, animal hormones, and antibiotics.

In an embodiment, the medical system can comprise a food scale e.g. a smart utensil can use an inertial sensor, accelerometer, or strain gauge to estimate the weight of the food-carrying end of utensil. In an embodiment, the medical system can comprise motion sensors used to detect food consumption; said sensors can be worn on a person's wrist, hand, arm, or finger. A smart watch, fitness watch, watch phone, smart ring, or smart bracelet can measure the speed, pace, or rate at which a person brings food up to their mouth while eating and provide feedback to the person to encourage them to eat slower if the speed, pace, or rate is high.

In various examples, a food-consumption monitor or food-identifying sensor can be selected from the group consisting of: receptor-based sensor, enzyme-based sensor, reagent based sensor, antibody-based receptor, biochemical sensor, membrane sensor, pH level sensor, osmolality sensor, nucleic acid-based sensor, or DNA/RNA-based sensor; a biomimetic sensor (such as an artificial taste bud or an artificial olfactory sensor), a chemiresistor, a chemoreceptor sensor, a electrochemical sensor, an electroosmotic sensor, an electrophoresis sensor, or an electroporation sensor; a specific nutrient sensor (such as a glucose sensor, a cholesterol sensor, a fat sensor, a protein-based sensor, or an amino acid sensor); a color sensor, a colorimetric sensor, a photochemical sensor, a chemiluminescence sensor, a fluorescence sensor, a chromatography sensor (such as an analytical chromatography sensor, a liquid chromatography sensor, or a gas chromatography sensor), a spectrometry sensor (such as a mass spectrometry sensor), a spectrophotometer sensor, a spectral analysis sensor, or a spectroscopy sensor (such as a near-infrared spectroscopy sensor); and a laboratory-on-a-chip or micro cantilever sensor.

A hand-held component comprising food-consumption monitor or food-identifying sensor can be selected from the group consisting of: smart utensil, smart spoon, smart fork, smart food probe, smart bowl, smart chop stick, smart dish, smart glass, smart plate, electronically-functional utensil, electronically-functional spoon, electronically-functional fork, electronically-functional food probe, electronically-functional bowl, electronically-functional chop stick, electronically-functional dish, electronically-functional glass, electronically-functional plate, smart phone, electronic tablet, and digital camera.

In an example, the volume of food consumed can be estimated by analyzing one or more pictures of that food. In an example, volume estimation can include the use of a physical or virtual fiduciary marker or object of known size for estimating the size of a portion of food. Volume can be estimated by using a device projecting (laser) light points or known grid onto food (measuring image deformations).

Geolocation can be used to refine probabilities of consumption (e.g. in a restaurant, a user is likely to eat some food of possibly published menus). Conversational devices can ask a person clarifying questions concerning food consumed. In some embodiments, the medical system can comprise a human-to-computer interface for entering information concerning food consumption. Such a system can comprise a microphone, speech recognition, and/or voice recognition interface; touch screen, touch pad, keypad, keyboard, buttons, or other touch-based interface; camera, motion recognition, gesture recognition, eye motion tracking, or other motion detection interface; interactive food-identification menu with food pictures and names; and interactive food-identification search box.

### Glucose Sensor

In some embodiments, the measured analyte can be blood and/or interstitial glucose.

Along/aside glucose, many other blood/body analyte can be measured.

The “analyte sensor” or “sensor” or “sensor electronics unit” can be an implantable glucose sensor, or a transcutaneous glucose sensor, or a dual electrode analyte sensor.

The glucose sensor can be configured to measure in vivo a signal indicative of a glucose concentration. The sensor can comprise one or more electrodes (for example made of metal oxide, one or more electroactive surface, one or more biocompatible membranes configured to reduce a flux of glucose there through). The sensor electronics can be configured to process the signal from the sensor.

The sensor can comprise a membrane impregnated with an oxidase, a bioprotective membrane substantially impermeable to macrophages and an angiogenic layer. The sensor can be a subcutaneously implantable enzymatic sensor (e.g. enzyme-catalyzed oxidation of glucose to gluconic acid and hydrogen peroxide, the latter being monitored amperometrically by the sensor). In an embodiment, the sensor comprises an electrically conductive noble metal (e.g. platinum or platinum-iridium) electrode covered with electrically insulative material, with a portion of this material removed from the electrode to define an enzyme-receiving zone (e.g. a short length of polytetrafluoroethylene coated platinum-iridium wire presenting a protrusion or recession enzyme-receiving zone. An enzyme can be operably immobilized on an exposed section of the platinum-iridium wire, for example by an adsorption of the enzyme on a cellulose acetate or Nafion layer followed by cross linking with glutaraldehyde.

In an embodiment, a synthetic polymer membrane disposed over the enzymatic indicating surface can serve as a permeable protective layer (e.g. polyurethane, thickness of from about 5 to 10 microns) as well as a diffusional barrier for glucose which slows down the flow of glucose and creates a linear sensor response over the concentration ranges of interest. In an embodiment, the use of an additional, negatively charged inner membrane layer immediately adjacent the Pt-Ir wire can retard the diffusion of negatively charged species or interfering species (e.g. ascorbate and urate), while said inner membrane does not significantly exclude hydrogen peroxide and electrically neutral species.

In some embodiments, reagents can comprise glucose oxidase (glucose or glucose oxidase in Bovine Serum Albumin (BSA). Sensor material can comprise platinum and/or silver (or gold/chrome on polyimide base)

In some embodiments, the invention uses invasive and/or non-invasive and/or minimally invasive glucose or blood analyte' measurement devices. In an embodiment, the patient can wear one or more contact lenses configured to measure or estimate BG values.

The glucose sensor and/or the extension can be further miniaturized, to microscopic scales (if not to nanoscopic scales). A plurality of said sensors and/or extensions can be distributed on the body of the patient. The extension can be mobile to some extent around the glucose sensor (e.g. can be rotated around the sensor's pivot). In an embodiment, the glucose sensor is reusable and can move along a belt worn by the patient (for example around the belly or around the wrist in case of a miniaturized embodiment).

In some embodiments, a plurality of glucose sensors and/or or extensions can be used, for example in parallel. One glucose sensor can be associated with one extension. A plurality of glucose sensors can be associated with one extension. A plurality of extensions can be associated with one glucose sensor. A plurality of glucose sensors can be associated with a plurality of extensions. Such embodiments advantageously can increase the reliability of measures and/or the robustness of the global system (depending on which one is determined to be the weak part of the chain)

A sprinkler sensor can be used (e.g. with different openings to capture different analyte at different skin depths)

### Drug Delivery Device

In some embodiments of the medical system according to the invention, at least one actuator is a drug delivery device.

In particular, the actuator can be a pump. The drug can be insulin.

Along/aside insulin, many other drugs can be injected or otherwise be made available or accessible.

An injection device can be passive, e.g. operated manually. An injection device can be operated locally and/or at distance; for example a remote injection device can be triggered remotely by a doctor to a patient provided with an injection device. Local and remote commands can cooperate (e.g. with predefined cooperation and/or synchronization rules). Optional authentication mechanisms can be configured and further drug delivery can be conditionally authorized.

In some embodiments, the drug administration pump is modular. Interconnectable modules can be assembled to get a scalable diabetes management system. For example, a smartphone can comprise a plurality of slots or bricks or modules, each serving a dedicated function, either for general computing or IT purposes (CPU, memory, telecommunication, energy or battery) or for medical purposes (analyte test strip slot and reader, DNA sequencer and/or synthetizer, microfluidics circuit, drug administration, etc). Medical and non-medical services can be separated with intention or integrated (to some predefined and controlled extent). For example, power management can prioritize between critical medical processes and non-critical (medical or non-medical) services (e.g. computing power used by gaming applications). Power management can occur at software (according to different granularity levels, ranging from apps to software processes if not threads)) and/or at hardware level (non-critical circuits or parts of circuits can be powered off or hibernated, etc).

### CGM/FGM

In some embodiments, the medical system according to the invention can comprise a continuous glucose monitoring sensor (CGM) and/or a flash glucose monitoring sensor (FGM). The sensor can be part of a CGM device.

In some embodiments, the medical system can comprise a flash glucose monitoring device associated with an electronic circuit configured to receive and/or send data to/from said flash glucose monitoring device and to/from a remote computer device such as a smartphone.

In some embodiments, glucose can be monitored via a monitoring watch, for example based on reverse iontophoresis (optionally adding mechanical vibration to flex the “patch” and enhance permeation, and also measure a ratio of sodium ions extracted along with the glucose to compensate for variations in flow).

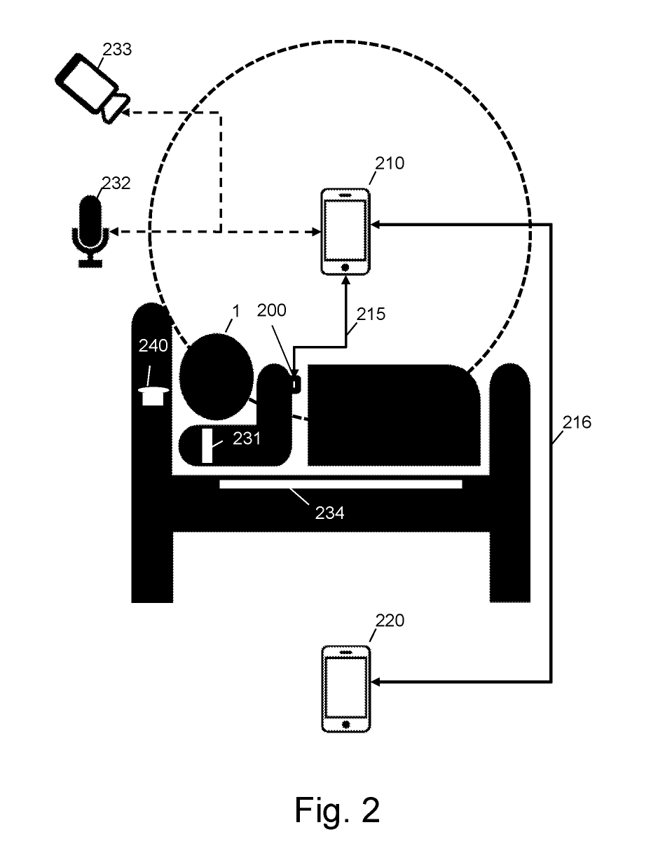

FIG. 2 shows a specific embodiment of the invention.

In an embodiment, a child 1 (more generally a “patient” or “user”) sleeping or resting on a bed is wearing a medical system 100 according to the invention. A first electronic device e.g. a smartphone 210 (at close proximity of the bed e.g. within NFC range 115) can query the medical system 100 and, in response to said query, can receive 215 data including a BG measurement value (for example as determined by a CGM and/or FGM device 200). The smartphone can communicate 216 (e.g. by Bluetooth Low Energy BLE and/or Wifi and/or mobile communication) said data including the BG measurement value to a second smartphone 220, for example located in the parents' sleeping room. The second smartphone 220 can execute a software application “app” which can handle BG values over time and possibly raise (e.g. audio) alarms, in order to wake up parents (for example in case of hypoglycemia).

In an embodiment, a software application (“app”) can be executed on parent's device P 220 and on room/child's device K 210. Both devices P and K can have been previously paired (e.g. by PIN code and/or passphrase and/or token) or be paired on-the-fly or an-demand.

The app K can receive data with/by/through a communication relay 240 (e.g. a BLE-Wifi bridge or a device configure for direct NFC readouts, etc). Data can be formatted (locally and/or on-the-fly and/or in the cloud and/or in app P). App K can send data to app P (for example by same Wifi and/or by SMS and/or by 3G/4G/5G networks). Data communications can be encrypted.

In addition to an invasive CGM and/or or minimally-invasive FGM, the apps K and/or P optionally also can receive other data from other devices.

For example, data can stem from a wristband monitoring heart rate 231, a microphone or baby phone 232, a video stream from a camera 233, a mattress 234 and/or other devices (not shown).

A heart rate monitoring wristband 231 can comprise an embedded oximeter and/or accelerometer. Heart rate zones can be configured and transmitted downstream to alert of too low or too high heart rates (hypoglycemia is at least significantly correlated with high hear rates).

In addition or in substitution, an audio baby monitor 132 can transmit (for example by DECT or by CPL) audio signals stemming from the child. A child enduring a hypoglycemia can convulse (make noise) or a contrario be abnormally silent. In case of the connection between smartphones 110 and 120 being interrupted, the audio channel can provide valuable information. Optionally, audio threshold can be configured to raise appropriate audio alarms (e.g. beyond normal breathing). The audio device 232 for example can be configured to transmit sounds of the child to parents above a configurable particular audio level (or a range of thresholds), possibly indicative of a suffering child in hypoglycemia, e.g. deeply breathing.

In addition or in substitution, a video camera 233 can detect movements in excess of one or more predefined thresholds and communicate an alarm to the smartphone 110. For example such a camera can monitor and quantify movements of the child, detecting abnormal gestures such as convulsions, or analyzing the color spectrum of the skin child, variations thereof being possibly associatable with heart rate.

In addition or in substitution, a breathing detector 234 (for example placed in/under the mattress) can monitor the breathing of the child and for example trigger an alarm in the absence of a detected breath over a predefined time interval and/or excessive breathes. Examples of respiratory sensors can comprise abdominal inductance bands, thoracic inductance bands, a non-contact bio motion sensor, or an airflow sensor. The monitored respiration parameters can include respiratory effort, respiratory movement, tidal volume, or respiratory rate.

In addition or in substitution, a “panic” button 240 can enable the child to trigger an audio alarm (a child in hypoglycemia may not be able to speak and a fortiori to shout for help).

The mentioned sensors/devices can cooperate (for example detecting sweat along a high heart rate can be indicative of an increase probability of a hypoglycemia event). The combination of sensor data can reveal more than the aggregation of signals. Readily available electronic consumer devices can advantageously be combined to provide a sensitive and robust integrated medical system. Sensors can be customized to the targeted tasks (for example a directional microphone can focus on capturing breathing sounds, a plurality of wristbands can be used in arms and legs, along oximetry devices, a T-shirt or belt or stretchable electronics can monitor displacements of the breathing body). In the software management layer, computer learning (for example deep learning) can fine tune the orchestration of sensors and/or actuators).

App P can receive data from app K (bidirectional communications are possible). App P can implement a diversity of hypoglycemia prevention algorithms. One or more algorithms for example can be downloadable from the Cloud and can be configured to analyze received BG data. Algorithms can be independently and/or concurrently and/or adversely performed. One or more algorithms can provide trend and/or target/time interval prediction and/or probability threshold. App P can be configured to emit alarms for example based on rules applied on data or facts (speaks up, TTS/audio alarm). App P can be provided with superadmin privileges. App P can setup personalized ranges (BG min, BG max, heart rate HR min, HR max, time intervals e.g. at 3 am). App P can setup personalized (easy and meta) alarm rules (for example “if HR>160 AND algorithm 1 prediction under 70 mg in 30 minutes then . . . ”; “if algorithm 1 and algorithm 2 difference>20% then ignore . . . ”). In an embodiment, rules are shared and commented online (“if this then that”).

Parents can setup the different configuration parameters and thresholds of the medical system according to the invention. False positives (wrong hypoglycemia alerts as determined by the medical system) generally do not constitute a problem, since parents highly welcome computer-assisted systems to be wakened up at night. It is preferable to be awakened for nothing than to miss a possibly severe hypoglycemia (and to rely by the mere transmission of natural sounds from a bedroom to another with no audio amplification at all).

More generally, beyond the instrumentation of the bedroom, the living place can be instrumented with various sensors, for example with tags (e.g. RFID tags embedded in the environment, e.g. doors of the apartment, door of the car, in the steering wheel, in the office, etc), said tags triggering reminders and/or measures. Logical rules also can be used (e.g. geofencing), in complement or in substitution of the instrumentation of the environment. For example, one or more (active and/or passive) RFID tags can be used. The spatial environment can be “coded” or “enriched” via NFC or RFID or other tags. When passing by, the RFID reader embedded according to the invention for example can read such tags distributed in the environment and following, this can trigger some specific and predefined actions. For example, in the bathroom, some appropriately positioned RFIDs tags can trigger an invitation to test blood glucose and/or a direct capture data stored in an FGM.

NFC generally operates at slow speeds, but an NFC tag advantageously does not require power, and generally doesn't require pairing. With NFC, the connection between two NFC enabled devices is automatically established in less than a fraction of a second. The maximum data transfer rate of NFC (424 Kbit/s) is slower than the one of Bluetooth V2.1 (2.1 Mbit/s). With a maximum working distance of less than 20 cm, NFC has a shorter range, which advantageously reduces the likelihood of unwanted interception. NFC is generally compatible with existing passive RFID (13.56 MHz ISO/IEC 18000-3) infrastructures. NFC Tags are an application of RFID technology. Unlike most RFID, which makes an effort to give a long reading range, NFC deliberately limits this range to only a few inches or almost deliberately touching the phone to the tag. In addition, some authentication can be added on top of the use of NFC tags (irreversibility also can be managed, with frangible/tearable connections).

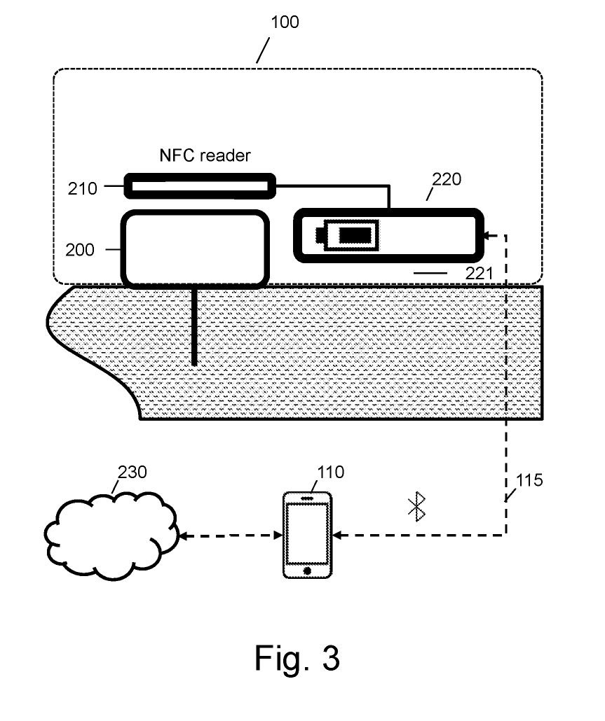

FIG. 3 shows another example of an embodiment of the invention.

In the described embodiment, the medical device 100 according to the invention comprises a FGM/CGM device 200.

The device 200 for example comprises a minimally invasive BG device 300, a reader 310 associated to an energy source and communication module 320, said module being releasable or attachable 321 to the body of the child.

In an embodiment, the FGM device 300 is NFC enabled, the reader 310 can be a NFC reader and the module 320 can be a Bluetooth Low Energy (BLE) module. Such combination of communication protocols correspond to advantageous compromises in terms of reliability, data transfer rates and energy consumptions.

The association of the assembly 200 to the body of the patient can be made in different manners 221. It can be releasable (e.g. glue and/or magnetic and/or plug and/or cradle, and/or Velcro and/or Gecko-based association, etc) or affixed (e.g. glue, melted, etc.)

The arrangement of elements 300, 310 and 320 can be made in various ways. The reader 310 is generally mounted on top of the CGM/FGM 300, but in some embodiments wave guides can be used and adjacent arrangements are possible. Wired connections are possible, but wireless inter-connections also can be implemented or both. In some embodiments, elements 300, 310 and 320 are natively integrated.

The communication channel 215 from device 200 to other devices (e.g. smartphones 210/220) can be wireless (e.g. BLE, Wi-Fi, Li-Fi, NFC, beacon, etc) and/or wired (e.g. rigid, flexible, releasable, magnetic, spring-based, torsion-able, etc). Wired communications are advantageous to avoid eavesdropping, interception, spoofing and other attacks (insulin delivery attacks can be prone to cyber-attacks). Encryption mechanisms can be used on top of transportation layers.

Once the data stream is enabled, further data processing can be performed (for example in the elastic “Cloud” 330), where algorithms can analyze and process data, in addition or in complement to smartphones 210/220.

With a Flash glucose measurement FGM device presents pros and cons, a patient can know BG values upon request (a scan gesture to trigger the retrieval of BG values is simple and fast to execute, as would be a glance at a CGM display device, thereby equating this class of devices in terms of user's attention). Incidentally, energy management can be optimized. A significant disadvantage of a FGM lies in the absence of continuous monitoring, i.e. the inability of raise alarms (in case of too high and/or too low values, but also in case of defavorable trends). According to the invention (described assembly), a FGM device 300 can advantageously be converted into a CGM device (and in a reversible manner).

Adding an extension repeating NFC measures advantageously allows detecting a possible malfunction of the glucose sensor, for example earlier than a manual request would have led to. A malfunction can comprise one or more of a problem with the patient tissue (e.g. occlusion, the sensor being pulled out or pulled off, etc), a hardware problem (e.g. leak in watertight seal, battery dysfunction, etc) and/or a software problem (e.g. a malware or malicious software executed by the hardware electronics of the glucose sensor, an abnormal drift in BG values, etc). In other words, the assembly according to the invention (extension 310 and 320 to FGM 300) can serve as a watchdog or a monitoring device to monitor the glucose sensor itself. As the extension according to the invention can embed more expensive electronics than the disposable sensor, a valuable combination of a sensor with an additional hardware can be conceived. In some embodiments, the cost of the disposable sensor can be even further reduced by deporting more expensive parts into the extension. In some embodiments, more than two parts (i.e. sensor and extension) can be used: an N-tier architecture can advantageously support various advantages, in terms of medical value, computer security, and business models.

In an embodiment, the medical system comprises a glucose sensor GS configured to communicate one or more interstitial blood glucose IBG values by near field communication; a hardware bridge circuit HBC, comprising an NFC reader and configured to read IBG values from said glucose sensor and to communicate said IBG to a computer. Advantageously, without the HBC, the medical system can function as an on-demand glucometer (type 1 and type 2 diabetes). With the releasable HBC, the system can be a full featured CGM (T1D). In an embodiment, an adjacent or integrated mechanical arrangement does not increase the thickness of the medical system under clothes. In an embodiment, the HBC is adapted to filter IBG values before communication. The HBC can be an active device, beyond a passive relay: it for example can implement at least some hypo detection algorithms. Remotely accessed data processing resources can allow for more computer power and flexibility. With elastic processing means, FDA-approved algorithms can handle anonymized IBG data and return hypo predictions. In an embodiment, the GS can be configured to last or remain 15 days in an (subcutaneous) inserted state.

FIG. 4 illustrates association schemes of sensors and/or actuators according to embodiments of the invention.

In an embodiment, a plurality of sensors, actuators or devices can be used in combination. The cooperation or orchestration of such pluralities of sensors, actuators or devices can be performed in various ways, possibly dynamically (e.g. adaptatively). A diversity of medical (e.g. diabetes) management regulation schemes can envisioned, based on such combinations.

In graph theory, a graph, or set of “vertices” (or “nodes”, or “points”) connected by “edges” (or “arcs” or “lines” or “arrows”). In a directed graph, the edges have a direction associated with them. In a mixed graph, some edges are undirected while some others are directed.

According to the invention, there is determined a graph defining the relations between devices (for example used for diabetes management).

Life with diabetes can require to continuously monitor/discover available devices and available stocks of accessible carbohydrates (“carbs”) and injectable insulin. Depending on life events, parts of required or advantageous devices can be unavailable (e.g. forgotten, broken, not immediately accessible because left in another room, etc). Combined with embodiments of the invention, available devices for diabetes management (e.g. sensing devices of any physiological indicator, insulin sources, ingestible carbohydrates in one form or another . . . ) can be discovered in the vicinity of the patient (by scans, geolocation history, machine vision, etc) and a diabetes management tactics (within the predefined global strategy associated with the patient) can be determined on a case-by-case basis, i.e. adaptively in real-time.

For example, a connected insulin pen can be determined as being available somewhere in the room at close proximity of the patient, while the fridge or source of sugar can be determined as being inferior to a particular distance thereby allowing carbs intake if needed. The possible presence of a connected television can then enable the opportunistic display of information indicating a dangerous low BG value and inviting the user to consider carbs intake or to compensate with an injection with said pen located in a radar view for example. If a pen is out of reach, then the assisting diabetes management system can suggest avoiding carbs for some time. An assisting diabetes software agent can be connected to a robot and/or a drone, enabling to reconfigure the environment for the patient and the specific current context (e.g. manage stocks preventively, for example insulin pens and/or sensors, determine recovery plans, etc). Such a holistic approach, partially automating user's behavior, is advantageous in that it allows the user to forget at best his disease, or at least to assist him in everyday life. Ever-changing contexts and the integration of a diversity of technologies can render this technical regulation task quite complex.

In an embodiment, there is disclosed a diabetes management system comprising on or more of interacting devices (“nodes” Ni of the regulation graph): an invasive sensing unit (N1), e.g. a blood analyte sensing unit (for example a FGM or CGM device, a glucometer, etc), providing essential data for therapy;—a drug delivery unit (N2), e.g. an insulin and/or glucagon pump, etc), allowing essential medical regulation;—a remote controller (N3), e.g. smartphone and/or smartwatches and/or glasses and/or Head-mounted displays HMD, etc), for display and/or entry of orders or commands, allowing man-machine interface;—a non-invasive sensing unit (N4) for example aggregating data from a plurality of sensors (e.g. environmental sensors such as ambient audio levels and/or physiological sensors e.g. a heart rate tracker), providing complimentary or optional data, and other devices (complimentary displays in the vicinity of the user, cloud computing resources, insulin pens for opportunistic injections, means for injection of other hormones, massage devices, etc),

At a fundamental level, two types of relations or relationships between nodes can be determined (“edges” of the graph), i.e. “interact with” and “control”. The verb “interact” designates a bidirectional relationship. The verb “control” means a unidirectional relationship. The verb “interact” is associated with (e.g. can be replaced by) verbs such as retroacts on, respond to, collaborate, cooperate, merge, relate, join, unite, interface, interplay, inter-react, co-act, concur, work with, participate, co-function or coordinate. The verb “control” is associated with (e.g. can be replaced by) verbs such as administer, manage, conduct, direct, execute, govern, head, pull, push, trigger, run, supervise, guide, regulate, order, command, dominate, influence, master, power or rule.

The expression “node N1 controls node N2” (active form) means that “node N2 is controlled by node N1” (passive form).

In some embodiment, diabetes management involves only two units X and Y, chosen from N1, N2, N3 and N4.

In a graph with 2 nodes and 2 types of edges, the following theoretical and distinct embodiments can be described. X interacts with Y. X controls Y. Y controls X. In a case wherein X is N1, Y is N2 and Z is N3, there are three possible embodiments: (1) the invasive sensing unit interacts with the drug delivery unit (both exchange data and commands, i.e. the insulin pump sends data back to the invasive sensing unit, for example by sending a confirmation command or by communicating a value of insulin bolus having been delivered, for example as determined by a flow sensor, or by sending an information indicative of an incomplete delivery or of bolus delivery speed, etc); (2) the invasive sensing unit controls the drug delivery unit (e.g. triggers an injection, for example as a “master-slave” configuration, with no retroaction or not data back from the insulin pump) and (3) the drug delivery unit controls the invasive sensing unit (e.g. triggers measurement upon occlusion, configures thresholds or ranges of thresholds in measurements by the sensing unit, etc).

FIG. 4 illustrates some specific embodiments with 3 or 4 nodes.

In some embodiment, diabetes/medical management can involve three units X, Y and Z, chosen from N1, N2, N3 and N4 (i.e. XYZ can be N1N2N3 or N2N3N3 or N4N2N1, etc). In a graph with 3 nodes and 2 types of edges, the following embodiments can be described (3 couples X-Y, Y-Z and X-Z, times 4 edge types i.e. “no relationship”, “interact” or “control” or “is controlled by”). Embodiments comprise: X controls Y, and X controls Z (“fan” configuration); X controls Y, and Y controls Z, and X controls Z (“feedforward loop” graph); X is controlled by Y, and Y controls Z, and X is controlled by Z (“feedback loop” graph); X interacts with Y, and Y interacts with Z, and X is controlled by Z (“feedback loop with two mutual dyads” graph) and X interacts with Y, and Y interacts with Z, and X interacts with Z (“fully connected” graph)

More generally, other embodiments comprise: X interacts with Y, and Y interacts with Z, and X interacts with Z; X controls Y, and Y interacts with Z, and X interacts with Z; X is controlled by Y, and Y interacts with Z, and X interacts with Z; X interacts with Y, and Y controls Z, and X interacts with Z; X interacts with Y, and Y is controlled by Z, and X interacts with Z; X interacts with Y, and Y interacts with Z, and X controls Z; X interacts with Y, and Y interacts with Z, and X is controlled by Z; X controls Y, and Y controls Z, and X interacts with Z; X is controlled by Y, and Y controls Z, and X interacts with Z; X controls Y, and Y is controlled by Z, and X interacts with Z; X interacts with Y, and Y controls Z, and X controls Z; X interacts with Y, and Y is controlled by Z, and X controls Z; X interacts with Y, and Y controls Z, and X is controlled by Z; X controls Y, and Y interacts with Z, and X controls Z; X is controlled by Y, and Y interacts with Z, and X controls Z; X controls Y, and Y interacts with Z, and X is controlled by Z; X controls Y, and Y controls Z, and X controls Z; X is controlled by Y, and Y is controlled by Z, and X is controlled by Z; X is controlled by Y, and Y controls Z, and X controls Z; X controls Y, and Y is controlled by Z, and X controls Z; X controls Y, and Y controls Z, and X is controlled by Z; Y interacts with Z, and X interacts with Z; Y controls Z, and X interacts with Z; Y is controlled by Z, and X interacts with Z; Y interacts with Z, and X is controlled by Z; Y interacts with Z, and X controls Z; Y interacts with Z, and X is controlled by Z; Y controls Z, and X controls Z; Y is controlled by Z, and X controls Z; Y controls Z, and X is controlled by Z; X interacts with Y, and X interacts with Z; X controls Y, and X interacts with Z; X is controlled by Y, and X interacts with Z; X interacts with Y, and X controls Z; X interacts with Y, and X is controlled by Z; X controls Y, and X controls Z; X is controlled by Y, and X controls Z; X controls Y, and X is controlled by Z; X interacts with Y, and Y interacts with Z; X controls Y, and Y interacts with Z; X is controlled by Y, and Y interacts with Z; X interacts with Y, and Y controls Z; X interacts with Y, and Y is controlled by Z; X controls Y, and Y controls Z; X is controlled by Y, and Y controls Z; X controls Y, and Y is controlled by Z.

In a specific example, wherein X is a medical system 200 according to the invention (comprising a FGM flash glucose monitoring device or sensor 300), wherein Y is a smartphone or computer system and Z is an actuator e.g. an insulin pump, the following embodiments can be described:

In the “fan” configuration, the flash glucose monitoring device controls the smartphone, and the flash glucose monitoring device controls the insulin pump; in other words, the smartphone acts as an intermediary to escape/transmit data but is not actively involved in the regulation.

In the “feedforward loop” configuration, the flash glucose monitoring device controls the smartphone, and the smartphone controls the insulin pump, and the flash glucose monitoring device controls the insulin pump; in other words, the smartphone acts as an intermediary which now can have some action on delivery as well, the insulin pump being controlled by both flash glucose monitoring device and the smartphone; this for example means that primary commands by the flash glucose monitoring device can be modulated or otherwise modified by the smartphone, for example knowing diabetes management rules);

In the “feedback loop” configuration, the flash glucose monitoring device is controlled by the smartphone, and the smartphone controls the insulin pump, and the flash glucose monitoring device is controlled by the insulin pump; in other words, “intelligence” is distributed in a different ways, which can lead to different robustness models.

In the “feedback loop with two mutual dyads” configuration, the flash glucose monitoring device interacts with the smartphone, and the smartphone interacts with the insulin pump, and the flash glucose monitoring device is controlled by the smartphone.

In the “fully connected” configuration, the flash glucose monitoring device interacts with the smartphone, and the smartphone interacts with the insulin pump, and the flash glucose monitoring device interacts with the insulin pump.

In some embodiment, the medical management can involve four units or elements sensors/or actuators W, X, Y and Z, chosen from N1, N2, N3 and N4. Likewise, different schemes can be identified. For example, the medical system can comprise X controlling both Z and W (“bi-fan” configuration), while Y also can control Z and W; the medical system can comprise X controlling both Y and W, which in turn control Z (“bi-parallel” configuration), thereby enabling an indirect control of Z by X; the medical system can comprise X controlling both Y, Y controlling Z, Z controlling W, W controlling X (“feedback loop” configuration), thereby leading to a sequential configuration with a final looping; the medical system can comprise X be controlled by Y and W, Z interacting with both Y and Z (“uplinked with two mutual dyads” configuration), thereby leading to a particular indirect relationship between X and Z; the medical system can comprise X controlling both Y, Y controlling Z, Z interacting with W and W interacting with X (“feedback loop with two mutual dyads” configuration), thereby combining a sequential side and an interacting side; the medical system can comprise X interacting both with Y and W (“fully connected” configuration), along with Z (partially or entirely reachable to X through Y and/or W); In other configurations, additional direct links are possible. For example, in the “fully connected” configuration previously described, there can be added a direct control of X onto Z.

X, Y, W and Z can be permuted (one by one, two by two, and three by three) in all the examples discussed above. Each (generic) configuration can be specified (for example in diabetes management). Each configuration can present regulation advantages (which can be theoretically determined or evaluated with test/use cases). A medical system can implement one or more configurations over time. The triggering of topological changes can be determined due to/by medical reasons, contextual data, etc.

The term “controllability” designates the number of control points of a system and the degrees of freedom for controlling it (the extent to which it can be acted upon). A diabetes system comprising a high number of sensors (heart rate, temperature, humidity/wet/sweat/perspiration/transpiration, EEG, ECG, etc), each sensor input acting as a input or be the object of a retroaction when coupled to an injection system associated with a measurement system can result in a global system, which can be complex. In addition the perimetric definition of such a super-system can be variable over time and/or space (a patient retrieving an insulin pen in the car can be offered more diabetes management strategies). Such a complex super-system nevertheless can be “controlled” to some extent (e.g. be fault-tolerant, present redundancies, be sensitive or robust to erroneous if not falsified data, etc). Particular states of the global complex system may prove to be critical or unbalanced or imbalanced. Diabetes management rules can handle such flexible evolving system (e.g. adaptive system).

### Other Embodiments and Aspects of the Invention

The “internet of things” (IoT) or “pervasive computing” or “Web of Things” designate the network of physical devices, vehicles, buildings and other items—embedded with electronics, software, sensors, actuators, and network connectivity which enable these objects to collect and exchange data. In some embodiments, the medical system according to the invention for example can use techniques of the programmable Web (e.g., REST, HTTP, JSON), of the semantic Web (e.g., JSON-LD, Microdata, etc.), of the real-time Web (e.g., Websockets) and/or of the social Web (e.g., Oauth or social networks). IoT or WoT raise privacy and security concerns. To mitigate these risks, the medical system according to the invention can implement encryption mechanisms or privacy safeguarding mechanisms.

The medical system according to the invention can interact with the IoT. In an embodiment, the medical system according to the invention and/or at least parts of the IoT can be a non-deterministic and open network in which auto-organized or intelligent entities (Web services, SOA components), virtual objects (avatars) will be interoperable and able to act independently (pursuing their own objectives or shared ones) depending on the context, circumstances or environments. Among other properties, the medical system will feature an autonomous behavior (e.g. through the collection and reasoning of context information) interacting with the objects ability to detect changes in the environment (e.g. faults affecting sensors) and to introduce suitable mitigation. A human being, and the associated medical system, when placed in an urban environment, may be surrounded by 1000 to 5000 trackable objects in the near future: the medical system will intensely interact with its environment.

In some embodiments, one or more crypto ledgers (e.g. blockchain) can be used (to secure the archiving of data according to a trustless model). In some embodiments, hardware and software architecture according to the invention can use one or more secured medical crypto ledgers. For example, one or more crypto ledgers (e.g. blockchain) can be used, advantageously providing reliable timestamping of medical data. Trusted timestamping and/or trustless timestamping can be used. Secure mechanisms can be built on top of such blockchain. For example, proof-of-work mechanisms can secure drug delivery, by requiring some work from the service requester (hard or moderately hard work on the requester side but easy to check for the service provider). Proof-of-work (PoW) functions for example comprise integer square root modulo a large prime, Weaken Fiat-Shamir signatures, Ong-Schnorr-Shamir signature, Partial hash inversion, Hash sequences, Puzzles e.g. Diffie-Hellman-based puzzle, Mbound, Hokkaido, Cuckoo Cycle, Merkle tree based, Guided tour puzzle protocol. Other mechanisms such as “proof of space” or “proof of bandwidth” or “proof of ownership” (proving that specific data are held by the prover e.g. the patient) also can be advnatageoulsy used. In some embodiments, proof-of-stake (PoS) techniques can be used, in order to achieve distributed consensus (for example to determine a medical action). In some embodiments, PoW can be hybridized with PoS.

In some embodiments, medical “smart contracts” can be implemented So-called “smart contracts” designate computer protocols which can facilitate, verify, or enforce the negotiation or performance of a digital “contract”, or that make a contractual clause unnecessary. Smart contracts can model diabetes therapy, for example by modeling a collection of therapeutic measures, decisions and actions. Medical smart contracts can be made partially or fully self-executing, self-enforcing (e.g. continuous verification), or both. Smart contracts advantageously can “transaction” costs associated with contracting. Some medical conditions, such as diabetes, essentially involving automation/biological regulatory mechanisms) can be coded or modeled in terms of (e.g. competing) programs or program subroutines or “transactions” (e.g. programmable cooperating APIs and/or tokens exchanges for example). A collection of programs can be executed concurrently and one or more arbitrage mechanisms determining a best decision (e.g. emerging from said negotiations).

A plurality of business models (e.g. pay walls) can be implemented—also in combination—with the different embodiments of the invention. A given business model can require specific technical features, which can range from a loosely combination up to a deeply integrated with the described embodiments of the invention. In an embodiment, the glucose sensor is provided free of charge. In an embodiment, the glucose sensor is provided according to a freemium model (e.g. advanced measures or data such as confidence intervals can be provided for a fee). In an embodiment, the glucose sensor is provided for a subscription fee (weekly or monthly or trimester or semester or yearly subscription), in which situation a disfunctioning sensor is for example replaced for free. In an embodiment, the glucose sensor is provided for a pay-per-scan basis (a person with type 1 diabetes will scan more often than a person with type 2, yet economies of scale may be reached by using the same platform). In an embodiment, payments and/or reimbursements can be automatically triggered (for example by using micro-payments, Bitcoins or other crypto ledgers). In an embodiment, the business model can comprise ad-support. Adblockers can be used. In some embodiments, the display of advanced or premium data (e.g. analysis of raw data, medical advices, etc) can be unlocked if the patient watches an ad or commercial, attested by a click or other methods to secure the display of advertisements. Other models can comprise sponsoring.

The present document is not a medical advice. The present examples are described in way to optimize defensive publishing. The “skilled person” or reader of the present patent application is invited to further read and follow the various social accounts of the inventor, as well as any other publication mentioned therein. It is incentivized to combine any one of these posts or publications with the embodiments or features described in the present patent application.

When lists of elements are provided, it is intended that—even if forgotten i.e. not explicitly mentioned—, combinations of such elements are possible. The expression “and/or” is generally applicable to this entire document; for example a sentence such as “the device can comprise element A, element B and element C” should be interpreted as “the device can comprise element A and/or element B and/or element C”, i.e. the device comprises “A and B and C”, or “A and B”, or “A and C”, or “B and C”, “A”, or “B”, or “C”.

A plurality of “incentives to combine” is now provided. It is suggested to the reader or skilled person to combine any of the presently described embodiments with one another.

It is also suggested to combine described embodiments or combinations thereof with one or more of the following techniques or technologies or concepts or ideas or paradigms or states or the like: accelerometer, accidental, acoustic, adhesive, adjustable, algorithm, allergy, anxiety (quantification), arterial (monitoring), artificial (pancreas), asleep (evaluation), audio metering (stress), auditory, automatic, autonomic, barcode (disposables), biochips (e.g. implantable), biorhythm (e.g. structured testing), bone (e.g. discrete sound transmission), Braille (e.g. computerized display), brain (e.g. interface), cannula (e.g. sprinkler), cartridge, catheter, cerebral (e.g. monitoring), classifiers (images, big data), compensate, cycles, distribution, EEG, electrocardiography, electrodes, electroencephalograph, exhaled (evaluation of ketones), expandable (e.g. inflatable devices for haptics), feedback (e.g. regulation management), flow meter (e.g. in vessels, cannula tip, etc), friction, gastric (insulin vector), gloves (e.g. I/O user interaction), glucose, goggles (AR/VR), gyroscopes, headphones (e.g. ear buds to monitor heart rate), heating (e.g. insulin depots), holographic (e.g. displays), hub (e.g. personal assistants with synthetic voice to enounce glucose values or therapy events), hydration (e.g. warnings or recommendations), hyperactivity (e.g. alerts), hypothalamic, identification (or authentication of insulin pens or other diabetes management devices), incontinence (e.g. measure of glucose in fluids), indicators, indicia, induction, inhaled, inspired, intestinal (e.g. probes), lung, lymphatic, magnetic (e.g. measures or association schemes), massage (e.g. to facilitate diffusion of insulin depots), micro-needles (e.g. insulin delivery roller, capillary blood capture), modular (e.g. architecture of assembly), muscle, muscular, myogram, needle, neuroelectric, neuromuscular, noise, oesophageal, olfactory, ophthalmo, dynamometers, oral, oximeter, oxygen, pacemaker, patch, pattern, piercing (e.g. removable cannula, implantable devices), phase, portable, probes, protection, pulmonary, pulse, pump, radiation, reloadable, respiratory, saliva (e.g. measure of glucose, release of stored aromas to raise alerts), scan, secretion, sensitivity, sensory, similarity, skin, stethoscopes, stress, strip, suction, swallowing, sweat, synchronising, tachycardia, teeth, telemetry, temperature, terahertz, thoracic, threshold, tissue, toxic, transmitter, transplant, urine, vacuum, vessel, wristwatch, ventilated (e.g. anemometry analysis to cool down circuits or to facilitate drug diffusion), vertex, vertice (e.g. graph analysis), vibrate (e.g. for drug diffusion), vibratile, video (e.g. feedback), video-game (e.g. therapy education), viral, virtual, virtual reality, virtualized (e.g. sandboxed process, resource, etc), virus, vision (e.g. machine vision), visual (e.g. codes, etc), visualize (e.g. in 3D, immersive), VM (i.e; virtual machine, to sandbox and isolate critical hardware/software), voice (e.g. voice commands, voice recognition, along Optical Character Recognition), volatile (e.g. memory unit, for amnesic computer systems and privacy management), vote (e.g. triplication), VPN (e.g. computer security), VR (virtual reality and/or augmented reality and/or mixed reality), wallet (e.g. for transactions management), warrant (e.g. for smart contract management), watchdog (e.g. as a regulation tool, along self-healing or repairing systems), watermark (e.g. security mechanisms), wavelet (e.g. image compression), wearable (e.g. computers), web (e.g. portals), web-service (or APIs for mashups), weigh (e.g. scale enabled by deformations of a known deformable material on a touch screen), widget, Wifi, wiki (e.g. diabetes management rules), work (e.g. proof-of-work), wrapper (e.g. to interconnect databases), read/write rights management, X-ray, XML (e.g. interoperable format), zero-knowledge (e.g. systems), zoom (e.g. adaptive zoom as a function of glucose level, 4K (ultra-high definition, for realistic immersive environments), Ultra HD, 5G (ultra-high speed bandwidth), 6lowPan, ACR (Automatic Content Recognition, for example nature and volume amount of French fries in a plate, for example assessed by an illumination successively projected onto the plate/table and subtracting methods), AllJoyn (interoperability framework), AMOLED (color fidelity for carbs recognition), ANT+(fitness data exchange), Carplay (glucometer in car e.g. in steering wheel), CAS (Conditional Access Systems), Deep learning, DSRC (Dedicated Short Range Communications), DVB (data broadcast), e-ink (low energy display), FTTB or FTTH, GPU computing, If That Then That (workflow for connected devices), IPS (In-door Positioning Systems, for medical management), LBS (Location Based Services), LoRA (machine to machine network), 3D MIMO, PicoDLP, PND (Personal Navigation Device), GNSS device, quantum dots, RTLS (Realtime Locating Systems), SIP (telephony), SLI (Scalable Link Interface), Smart Metering (connected meters in the house for data processing and/or relay), SoC (System on Chip) and ToF (Time of Flight, e.g. cameras for 3D).

It is also suggested to combine described embodiments or combinations thereof with one or more of the following words, suggesting techniques or technologies or concepts or ideas or paradigms or states or the like: abdomen, abrasion (access to subcutaneous and capillary or interstitial blood), absorbent, acoustic (e.g. massage, analysis), acousto-optic, activity (monitoring), acupuncture, adaptive, adenoids, adequate, adhesive, adjustable, adrenals, alarms, alcohol, allergenic, allergy, amplifiers, angular, ankle, apnoea, applicator, arm (band), arms, arrays, arterial, artifacts, artificial, as, aseptic, assistance, attachments, attention, audible, audio-metering, auditory, automatic, autonomic, avatar, bags, balloon, barcode, barrel, belt (moving automated prick), biochips, bioelectric, biofeedback, biological, bio-potentials, biorhythm, bladder, blade, bladeless, body, bonding, bone, brachycardy, braille, brain, breakable, breast, breath, cable, calibration, caliper, camera, canal, cannula, cap, capacitive, capacity, capillary, caps, capsules, cardiac, cardiograms, cardiography, cardiovascular, cartilage, case, casing, catheter, cathode, cavities, cavity, central, cerebral, cervix, chairs, chamber, charts, check, checking, chemical, chest, circuit, circuitry, circulation, clamping, clamps, classifiers, clinical, clips, clock, clothes, co2, cocking, codes, cognitive, coherence, collect, collets, colon, column, combination, combination, compartment, compensate, complementary, complex, compliance, component, compressed, compression, concentration, condition, conductive, conduit, confocal, connected, connector, console, contact, container, content, continuous, contractility, contraction, control, conversion, cooling, cord, correct, correlation, cough, counter, coupler, cover, covering, create, cuff, current, curvature, curved, cutter, cutting, cuvette, cycle, cycle, cylindrical, cytochromes, defective, deficit, deflected, deliberate, delivery, dementia, demodulation, density, dental, dentistry, depression, depth, derivation, derivatives, derived, design, detachable, detection, detector, diagnostic, dialysis, diapers, diascopy, dilation, dilution, direct, directing, direction, disabling, discarded, discomfort, disconnecting, discriminating, disease, disorder, displacement, display, disposable, distal, distance, distances, distraction, distribution, divided, division, double, drift, drive, duct, dye, dysfunction, ear, ease, ectopic, eczema, EEG, efferent, effort, ejection, elastic, elasticity, elastin, elbow, electric, electrical, electricity, electro, electro auscultation, electrocardiograph, electrocardiography, electrochemical, electrode, electroencephalograph, electrolyte, electromagnetic, electromyography, electronic, electropalatography, element, emergency, EMG, emission, employing, encephalograms, encephalographic, endocrine, endoradiosonde, endotracheal, energy, engagement, enhanced, enhancement, ensure, entering, entire, entry, environment, enzyme, epidural, epilepsy, equipment, erectile, ergometry, ergonometric, estimate, evacuated, evaluating, evaluation, evaluations, event, events, evoked, examination, examining, excised, exercising, exhaled, exocrine, expandable, expansible, expansion, expel, expert, expiratory, expired, extend, extensible, extension, external, extracorporeal, extraction, extrasystoles, eye, eyes, faces, facilitate, facilitating, factor, failure, fall, fallopian, FALSE, fasteners, fat, fears, features, feedback, female, fibrillation, field, fields, films, filtering, filters, finger, fingerprinting, fingerprints, fingers, firing, fit, fittings, flaps, flicker, floating, floor, flow, flowmeter, flowmeters, fluid, fluids, fluorescence, foetal, foetuses, folded, foot, footprinting, footwear, for, force, forces, foreign, form, formed, forwarding, Fourier, fraction, fragility, free, frequency, friction, from, fully, function, functions, fundus, furniture, further, fusion, fuzzy, gain, gait, galvanic, games, garments, gas, gaseous, gases, gastric, gastro, gastrointestinal, gating, gauging, general, generated, generating, generation, geometric, geometry, glands, global, gloves, goggles, goniometers, GPS, graphical, graphics, grasping, grip, gripping, group, guide, guides, gums, gustatory, gynaecological, gyroscopes, haematocrit, haemodynamic, hair, hand, handling, hands, hardness, harness, harnesses, has, having, head, headphones, hearing, heart, heartbeats, heat, heated, heating, height, held, helical, helmets, high, higher, hip, holders, holding, holes, hollow, holographic, home, hospital, housing, housings, hub, human, hydration, hygienic, hyperactivity, hypodermic, hypothalamic, icons, identical, identification, identifying, image, imaging, immobilized, immune, impact, impaired, impedance, impede, implantations, implanted, implanting, impulses, incontinence, incorrectly, indentation, indexing, indicators, indicia, induced, inducing, induction, indwelling, inflamed, information, infra, infrared, infusion, inhaled, initiated, injection, injuries, input, inserted, inside, inspired, instance, instruments, insulating, integrally, integrated, integration, integumentary, intended, interactive, interchangeable, interest, interface, intermediate, internal, internet, interstitial, interval, intervals, intestinal, intestine, into, intolerance, intra, intracranial, introduced, introducing, introduction, invasive, invasively, investigating, involving, ionised, ions, irritation, is, isolated, isolation, items, its, jaw, joined, joining, joints, joystick, Kalman, keeping, keyboard, keyboards, kidney, kits, knee, knives, laboratory, lancet, lancets, lancing, laser, layer, leads, leaf, leaks, least, leaving, left, leg, length, lens, level, lids, lie, ligaments, light, like, limb, limbs, limited, limiting, line, linear, linked, lips, liquid, liquids, live, liver, load, loaded, lobe, local, located, locating, location, lock, locking, long, loop, loss, low, lower, Luer, lung, lymph, lymphatic, machine, magnetic, magnetism, magneto, magnetocardiographic, magnets, magnifying, main, maintaining, male, malignant, mammary, mammography, manual, manually, manufacture, manufacturing, mapping, markers, marking, marks, marrow, masks, masticatory, matching, material, mats, means, measured, measurement, measurements, measuring, mechanical, mechanism, mechanisms, media, medical, medication, medicinal, medicine, medium, melanin, membrane, memory, metabolism, method, methods, mice, micro, microdialysis, microneedles, microwaves, mind, minute, mirrors, missing, mixing, mobility, mode, modified, modular, modulation, module, modules, mole, monitoring, motion, motor, moulded, moulding, mounted, mouth, movable, movement, MRI, multiple, muscle, musculoskeletal, muscular, musculoskeletal, music, myogram, nail, nails, neck, needle, needles, negative, nerve, nerves, nervous, nesting, network, networks, neural, neuroelectric, neuromuscular, node, noise, non, nose, notification, nuclear, number, nystagmus, objective, obstetric, obtain, obtained, occluders, occurrence, occurring, oculography, oedema, oesophageal, oesophagus, office, olfaction, olfactory, onset, operation, operative, operators, ophthalmodynamometers, optical, opto, optocouplers, oral, order, organ, organs, oriental, originating, orthopaedic, oscillometric, oscilloscopes, output, ovaries, oxidase, oximeters, oxygen, pacemaker, pacemakers, packages, packaging, pain, palpation, pancreas, parallel, parameters, part, particle, particular, passageway, patches, patient, pattern, patterns, peak, penetration, perception, percutaneous, perforating, perforation, performing, period, periodic, peripheral, permanent, permanently, perpendicular, personal, personality, persons, PH, phase, photoacoustic, photographic, photometrical, photoplethysmograph, physical, physiological, pierce, pierceable, pierced, pierces, piercing, piezoelectric, piezos, pigtail, pile, pinion, piston, pistons, pitch, pituitary, pivotable, pivoted, placenta, plantar, plasters, playback, players, plethysmographic, plethysmography, plots, pneumatic, pneumography, podologic, pointed, pointing, polarisation, polarographic, portable, portion, ports, position, positioned, positioning, possible, posture, precedence, predetermined, predicting, pregnancy, preliminary, preparation, preparations, preparing, prescan, presence, pressing, pressure, pressurising, pressurized, prevent, preventing, prevention, printing, prion, private, probe, probes, procedure, processes, processing, produced, production, products, profile, progression, projections, promoting, propagation, propelling, properties, prospective, prostate, prosthesis, prosthetics, protection, protective, protectors, provided, providing, proximal, psychological, psychotechnics, pulling, pulmonary, pulse, pump, pumps, puncturing, purely, purposes, pushing, quality, quantity, rack, radial, radiation, radioactive, radiowaves, rails, Raman, range, rash, rate, ray, re, reaching, reaction, reactions, reader, ready, reagent, recognizing, recognition, reconstruction, recording, recover, red, reducing, reduction, reference, reflux, regardless, region, registration, regulating, rejection, relation, relay, release, reliability, reloadable, reloading, remaining, remote, removal, removed, removing, renal, rendering, repositioning, reproductive, resilient, resonance, respiratory, response, responsive, restraints, restricted, retention, retracted, retracting, retraction, retrospective, reusable, rigid, rings, risk, rod, roll, rolled, rolling, room, rooms, rotated, rotating, rotation, rotational, rubbing, ruler, running, rupturing, safety, saliva, sample, samples, sampling, scalp, scan, scanner, scanning, scar, screw, screwing, seal, sealable, sealing, sebum, secretion, secretions, security, seizure, selection, self, semi, seminal, sense, sensing, sensitive, sensitivity, sensor, sensory, sent, separate, separated, separately, separating, sepsis, serial, services, set, setting, sexual, shaft, shape, shaped, shapes, sheath, shielding, shields, short, shoulder, side, signal, signals, similarity, simulator, simultaneous, simultaneously, single, sit, site, sits, size, skills, skin, sleep, sleeve, slide, slides, sliding, slowly, small, smell, snap, soft, solenoids, soluble, sore, sound, sounds, source, space, spaced, special, specially, specific, spectra, spectral, spectroscopy, speech, speed, sperm, sphincters, spinal, spine, spleen, splines, sport, spring, stack, stacking, stage, stages, state, static, statistical, status, stents, steps, sterilization, stethoscopes, sticks, still, stimulation, stimulators, stimuli, stocked, stocking, stomach, stoppers, stops, strap, straps, stream, strength, stress, strip, structures, studies, subject, subjective, subject's, substance, substantial, subunits, such, suction, sufficient, superposing, support, supports, surface, surgical, sutures, swallowing, sweat, switching, symbols, synchronization, synchronising, synthesizing, syringe, syringes, system, tables, tablet, tachycardia, tactile, tailors, tampered, taste, technique, teeth, telemetry, telephone, telephones, temperature, tempered, template, templates, tendons, terahertz, terminating, test, testicles, testing, texture, than, that, the, their, then, therapeutic, therapy, thermal, thermo, thermometers, thickness, thoracic, threads, threshold, thresholds, thymus, thyroid, tight, time, tinnitus, tip, tissue, tissues, to, together, tomography, tongue, tonsils, tooth, toothed, topography, torque, torsion, total, touch, tourniquets, toxic, toxicology, toy, tracer, track, tracking, tracts, training, trajectory, transducer, transformer, transillumination, transition, transmission, transmitted, transmitter, transmitting, transplant, transplanted, trauma, travel, treatment, tremor, trends, tribometry, triggering, trunk, tube, tubing, tumescence, tumour, twinkling, tympanic, ulcer, ultrasound, umbilical, uncovering, unit, unsterile, urethral, urinary, urine, urological, uterine, uterus, vacuum, vagina, vaginal, validity, value, valve, variability, variable, variation, vector, vehicle, vein, velocity, venous, venting, ventricular, vesicles, vessel, vests, vibration, viscosity, vision, visual, visually, vital, vitality, vivo, voice, volume, walking, water, wave, waveform, wavelengths, wavelet, waves, welding, wheel, wheelchair, widening, window, wings, wireless, wires, wound, wrinkle, wrist, and wristwatch.

In an embodiment, the medical system comprises one or more sensors associated with one or more actuators. In an embodiment, the system can further or alternatively comprise one or more logic circuits configured to control and/or to interact with one or more of said sensors and/or actuators. In an embodiment, the system (of any one of the preceding embodiments, i.e. with or without logic circuits) can further or alternatively comprise one or more user interfaces. In an embodiment, parts of the medical system (of any one of the preceding embodiments) can be arranged and/or configured according to association schemes. In an embodiment, in addition or in substitution, the medical system (of any one of the preceding embodiments) or parts thereof can be arranged and/or configured according to one or more communication schemes. In an embodiment, in addition or in substitution, the medical system (of any one of the preceding embodiments) or parts thereof can be arranged and/or configured according to one or more security schemes. In an embodiment, in addition or in substitution, the medical system (of any one of the preceding embodiments) or parts thereof can be arranged and/or configured according to one or more cryptographic schemes. In an embodiment, in addition or in substitution, the medical system (of any one of the preceding embodiments), parts thereof and/or the control thereof can be arranged and/or configured according to one or more medical management rules. In an embodiment, in addition or in substitution, the medical system (of any one of the preceding embodiments), parts thereof and/or the control thereof can be arranged and/or configured according to one or more social mechanisms. In an embodiment, in addition or in substitution, the medical system (of any one of the preceding embodiments), parts thereof and/or the control thereof can be arranged and/or configured according to one or more energy management schemes. In an embodiment, in addition or in substitution, the medical system (of any one of the preceding embodiments), parts thereof and/or the control thereof can be arranged and/or configured according to one or more time and/or space schemes. In an embodiment, in addition or in substitution, the medical system (of any one of the preceding embodiments) can comprise at least one sensor for determining the concentration of an analyte and/or of a biomarker. In an embodiment, in addition or in substitution, at least one sensor can be minimally-invasive or non-invasive. In an embodiment, in addition or in substitution, at least one actuator is implementable. In an embodiment, in addition or in substitution, the medical system (of any one of the preceding embodiments) can comprise a contact lens and/or a spectrometer and/or a drone and/or a wearable computer. In an embodiment, the monitored analyte can be blood glucose. In an embodiment, the monitored analyte can be interstitial glucose. In an embodiment, in addition or in substitution, at least one actuator can be a drug delivery device. In an embodiment, the drug can be insulin. In an embodiment, in addition or in substitution, the medical system (of any one of the preceding embodiments) can comprise a continuous glucose monitoring sensor. In an embodiment, in addition or in substitution, the medical system (of any one of the preceding embodiments) can comprise a flash glucose monitoring device associated with an electronic circuit configured to receive and/or send data to/from said flash glucose monitoring device and to/from a remote computer device (such as a smartphone and/or a smart watch). Any one of the preceding embodiment can be combined with any one of other preceding embodiments. For example, the medical system can comprise one or more sensors associated with one or more actuators, and one or more logic circuits configured to control and/or to interact with one or more of said sensors and/or actuators, and one or more user interfaces as well. As another example, the medical system can comprise one or more sensors associated with one or more actuators, wherein parts of the medical system are arranged and/or configured according to security schemes, and wherein at least one sensor is minimally-invasive or non-invasive, and wherein the analyte is interstitial glucose.

1. A medical system comprising one or more sensors associated with one or more actuators.

2. The system of claim 1, further comprising one or more logic circuits configured to control and/or to interact with one or more of said sensors and/or actuators.

3. The system of claim 1, further comprising one or more user interfaces.

4. The system of claim 1, wherein parts of the medical system are arranged and/or configured according to association schemes.

5. The system of claim 1, wherein the medical system or parts thereof are arranged and/or configured according to one or more communication schemes.

6. The system of claim 1, wherein the medical system or parts thereof are arranged and/or configured according to one or more security schemes.

7. The system of claim 1, wherein the medical system or parts thereof are arranged and/or configured according to one or more cryptographic schemes.

8. The system of claim 1, wherein the medical system, parts thereof and/or the control thereof are arranged and/or configured according to one or more medical management rules.

9. The system of claim 1, wherein the medical system, parts thereof and/or the control thereof are arranged and/or configured according to one or more social mechanisms.

10. The system of claim 1, wherein the medical system, parts thereof and/or the control thereof are arranged and/or configured according to one or more energy management schemes.

11. The system of claim 1, wherein the medical system, parts thereof and/or the control thereof are arranged and/or configured according to one or more time and/or space schemes.

12. The system of claim 1, wherein at least one sensor determines the concentration of an analyte and/or of a biomarker.

13. The system of claim 1, wherein at least one sensor is minimally-invasive or non-invasive.

14. The system of claim 1, wherein at least one actuator is implementable.

15. The system of claim 1, comprising a contact lens and/or a spectrometer and/or a drone and/or a wearable computer.

16. The system of claim 12, wherein the analyte is blood and/or interstitial glucose.

17. The system of claim 1, wherein at least one actuator is a drug delivery device.

18. The system of claim 17, wherein the drug is insulin.

19. The system of claim 1, comprising a continuous glucose monitoring sensor.

20. The system of claim 1, comprising a flash glucose monitoring device associated with an electronic circuit configured to receive and/or send data to/from said flash glucose monitoring device and to/from a remote computer device such as a smartphone.
